(19) United States
(12) Patent Application Publication
(10) Pub. No.: US 2019/0300831 A1
(43) Pub. Date: Oct. 3, 2019

(54) BIOMATERIAL HAVING DECREASED SURFACE AREA, DEGRADABLE SCAFFOLDS OF SAME, AND METHODS OF MAKING

(71) Applicant: Cal Poly Corporation, San Luis Obispo, CA (US)

(72) Inventors: Nathaniel Martinez, San Luis Obispo, CA (US); Andres W. Martinez, San Luis Obispo, CA (US); Edward Brandon Strong, San Luis Obispo, CA (US)

(21) Appl. No.: 16/368,352

(22) Filed: Mar. 28, 2019

Related U.S. Application Data
(60) Provisional application No. 62/649,829, filed on Mar. 29, 2018.

(51) Int. Cl. C12M 3/00; C12M 3/06; C12N 11/02; C12N 5/00; C12M 1/00

(52) U.S. Cl. CPC C12M 21/08; C12M 23/16; C12N 11/02; C12M 23/30; C12M 23/34; C12M 23/26; C12M 23/28; C12N 5/0062

(57) ABSTRACT

Paper supports for fluid or cells having a decreased surface area are provided. Methods of producing such support comprise contacting the support with a composition comprising sodium periodate such that the surface area is decreased. The support or a portion thereof are reduced in surface area and the reduced portion or reduced entire support is degradable. The resulting paper support may also have hydrophilic channels and/or hydrophobic barriers that are also reduced in surface area. In embodiments the support may be a micropad or support for producing a tissue have desired structure. The support is miniaturized dialdehyde paper support that is malleable and retains its structure.

### CROSS REFERENCE TO RELATED APPLICATION

This application claims priority to previously filed and co-pending provisional application U.S. Ser. No. 62/649,829, filed Mar. 29, 2018, the contents of which are incorporated herein by reference in its entirety.

### STATEMENT REGARDING FEDERALLY SPONSORED RESEARCH OR DEVELOPMENT

This invention was made with Government support under grant no. DMR 1709740 awarded by National Science Foundation. The Government has certain rights in the invention.

### BACKGROUND

Microfluidic paper-based analytical devices (microPADs) are emerging as cost-effective and portable platforms for point-of-care assays. Since their introduction in 2007, paper-based fluidic devices (microPADs) have been explored extensively as platforms for point-of-care diagnostic tests and as tools for basic research and teaching.¹⁻¹⁰ MicroPADs have many attractive qualities such as low cost, small size, and the ability to operate without supporting equipment or sources of power.¹¹ MicroPADs are typically made by patterning paper with hydrophobic inks using one of several different printing techniques in order to define hydrophilic channels and test zones bounded by hydrophobic barriers.^{2,4} A fundamental limitation of microPAD fabrication is the imprecise nature of most methods for patterning paper. One common limitation to most methods of patterning paper is that the hydrophobic inks tend to diffuse horizontally in the paper and blur the patterns that are printed, therefore it can be difficult to produce patterns with dimensions smaller than 1 mm.¹² The ability to fabricate devices with higher-resolution patterns could enable new capabilities for microPADs, as this would allow for the fabrication of smaller devices with higher channel density, which in turn could process smaller volumes of sample in shorter amounts of time.

Furthermore, cell culture is an essential tool for research in biology and biomedical engineering as well as for the development, testing and production of countless commercial products including vaccines, small-molecule drugs, antibodies and proteins.[B4-6] Cell culture in the research laboratory is often performed in two dimensions on flat plastic dishes, however, cells are known to develop differently when cultured on rigid 2D substrates as compared to their native three-dimensional environments. [B7, 8] This observation gave rise to an interest in 3D cell culture, where cells are cultured in solid porous scaffolds or suspended in liquid. [7, 8, 3] Tissue engineering has the objective of creating living tissues and organs for research and medical applications, and relies on many of the same basic techniques as used in 3D cell culture. [B9-11]

Scaffolds for cell culture and tissue engineering provide structural support for cell attachment and tissue development.[B 12] They are an essential component for 3D cell culture and tissue engineering, and a vast amount of research on the development of scaffolds and the preparation of suitable biomaterials for making scaffolds has been carried out over the past thirty years. [B8, 9, 11-16] The results of this work have led to the development of many sophisticated scaffolds and significant advances in the field of tissue engineering. [B 12] While there are too many examples of biomaterials and approaches for making scaffolds to review in detail, a general trend is that the preparation of scaffolds require a significant investment of time and resources. [B 11] Many scaffolds are made from expensive biomaterials that are shaped into appropriate scaffolds through fabrication techniques that require specialized equipment, time and significant expertise.[B 11] This creates a barrier for researchers who may be interested in 3D cell culture or tissue culture, but who do not have the resources or expertise to produce the required scaffolds. Furthermore, most of the current scaffolds are produced in the form of small two-dimensional sheets and cannot be shaped easily into more complex 3D structures or patterned into distinct zones for culturing different types of cells on the same scaffold.[B 1-13]

### SUMMARY

The present methods provide a method to decrease the surface area of a paper support for fluid or cells or a portion thereof by contacting the support with a composition comprising sodium periodate for a period of time until the support surface area is decreased compared to a support not having been exposed to sodium periodate. The resulting support retains its structure but is malleable. Further, the support or portion thereof having been reduced in surface area is degradable, and in an embodiment is degradable in an aqueous solution. This allows a portion of the support or the entire support to be degraded. In an embodiment, where the entire support is contacted with the sodium periodate, cells may be deposited on the support and the underlying paper support then degraded to produce a desired cell formation and structure. An embodiment provides the composition comprising sodium periodate is contacted with the paper support until the percent oxidation of cellulose of the paper is 10% to 100%. In still additional embodiments, the composition comprising sodium periodate comprises at least 0.1M sodium periodate. When the composition is heated to 20° C. to 95° C. sodium periodate in excess of 1M can be produced. An embodiment provides concentration of 2.5M sodium periodate. The methods are useful for producing degradable miniaturized micropads/microfluidic devices. The produced support also provides for retained or even increased stabilization of proteins and/or nucleic acid molecules used in the support compared to a support not contacted with the sodium periodate. Further embodiments of the methods and resulting supports are provided.

### BRIEF DESCRIPTION OF THE DRAWINGS

FIG. 1 is a schematic showing oxidation of cellulose (FIG. 1(1)) with sodium periodate to generate 2,3-dialdehyde cellulose (FIG. 2(2)).

FIG. 2 is a diagram of a microPAD used for the glucose assay.

FIG. 3 shows a photograph A) displaying 78% reduction in surface area; a B) Scanning electron microscope (SEM) surface images of Whatman No. 1 chromatography (CHR) paper (400× magnification) where fibers appear to be more compact following miniaturization; and C) SEM cross-sectional images of Whatman No. 1 CHR paper (200× magnification). Miniaturized microPADs displayed 166% increase in cross-sectional width.

FIG. 4 are graphs showing miniaturization of microPADs over time. A) Plot of microPAD surface area versus reaction time for various concentrations of aqueous sodium periodate (NaIO4). B) Miniaturization comparisons of: non-patterned chromatography paper, microPADs with channel outlines (reduced wax), and microPADs with a full wax background (standard) in 0.5 M NaIO4. Non-patterned chromatography paper displayed the greatest amount of miniaturization, albeit to a minor degree. For both plots, data points represent the mean of three replicates, and error bars represent one standard deviation from the mean.

FIG. 5 shows characterization of miniaturized microPADs. A) Photograph of a functional microPAD pre- and post-miniaturization. B) Diagram of the device used for the minimum hydrophobic barrier test. A functional barrier prevented fluid from wicking into the empty zone (readout well) for at least 30 minutes. C) Diagram of the device used for the minimum hydrophilic channel test. Channel was determined to be functional if fluid could wick from the sample zone to the readout well. D) Summary data table comparing standard versus miniaturized microPADs. Miniaturized microPADs displayed a significant reduction in all characteristics except cross-sectional width. Values are given as the mean of all replicate measurements, +/− one standard deviation of the mean.

FIG. 6 is a graph of miniaturized microPAD surface area versus volume of 0.5-M NaIO4 used 4 for the miniaturization process. The initial surface area of all devices was 20.25 cm2. The height of the bars represents the mean of seven replicates and the error bars represent one standard deviation from the mean. These results indicate that a minimum of 0.30 mL 0.5-M NaIO4 per cm2 of microPAD is required for complete miniaturization.

FIG. 7 shows two graphs and a photograph demonstrating miniaturized microPADs as platforms for biochemical assays. A) is a graph showing the calibration curve as generated from a colorimetric paper-based glucose assay. The data was fit with a linear trendline. Data points represent the mean of eight replicates and error bars represent one standard deviation of the mean. B) is a graph showing the calibration curve for a colorimetric horseradish peroxidase (HRP) assay. The data was fit with a linear trendline. Data points represent a single replicate. C) is a photograph of the test zones after performing the HRP assays. Higher HRP concentrations produced higher color intensities in the test zones.

FIG. 8 shows results of paper treatment with sodium periodate. A) is an image of wax-patterned Whatman Grade 1 Chr paper before (left) and after (right) treatment with sodium periodate (scale bar=5 mm). B) is a graph of Whatman Grade 1 Chr paper surface area after treatment with various concentrations of sodium periodate over time. CO is an image showing enzymatic activity stabilization of HRP on MDAP over a period of 6 days. (TMB for 30 minutes).

FIG. 9 is an image of chromatography paper rolled into the shape of a tube (left) and MDAP rolled into the shape of a tube (right). (Scale bar=5 mm.)

FIG. 10 are images of A) a SEM image (100×) of a cross-section of chromatography paper; B) a SEM image (100×) of a cross-section of MDAP; and C) a SEM image (400×) of a cross-section of MDAP.

FIG. 11 is an image showing degradation of MDAP scaffolds in DMEM (left) and 1×PBS (center) over a period of 72 hrs. Chromatography paper was also tested as a control (right) (scale bar=1.5 cm).

FIG. 12 is a schematic diagram of plastic cassette with micro-channels and patterned platforms bathed in continuous flow of aqueous solutions.

FIG. 13 are images showing a 9-zone wax-patterned MDAP, immersed in 1×PBS over a period of 72 hrs (left). Enlargement of a single zone (right) showing the wax scaffold holds integrity while the MDAP zone has degraded.

FIG. 14 are images showing linear, monolayer growth of COS-1 fibroblasts (white arrows) along independent MDAP fibrils (black arrows) after 12 hrs incubation. (40× magnification).

FIG. 15 is a schematic diagram of the preparation of 3D tissues with different cell types. Growth factors can be deposited on patterned MDAP in individual layers. Different cell types can then be seeded and allowed to adhere to the scaffold layers. The individual layers of MDAP can then be stacked on top of each other to produce a 3D structure. As the MDAP scaffold degrades the different cell types can grow and fuse with each other into a single tissue stack.

FIG. 16 are schematic diagrams showing A) preparation of tissue-engineered blood vessels (TEBVs) from shaped MDAP; and B) a schematic of wax pattern for preparing patterned TEBVs.

FIG. 17 are images showing comparison of standard cellulose paper and miniaturized dialdehyde paper (MDAP). A) Photograph of standard (right) versus miniaturized (left) microPAD (approximately 78% reduction in surface area). B) SEM of top of normal cellulose (400× mag.) C) SEM of top of MDAP (400× mag.). D) SEM cross-section of normal cellulose (200× mag.). E) SEM cross-section of MDAP (200× mag.).

FIG. 18 are graphs showing spectroscopic characterization of MDAP. A) Infrared spectra of MDAP (dissolved 30 mg/ml in 1×PBS). B) Raman spectra of MDAP. Red circles indicate the presence of an aldehyde functional group.

FIG. 19 are graphs showing HRP and HRP-IgG stability curves.

FIG. 20 are graphs showing AP stability curves.

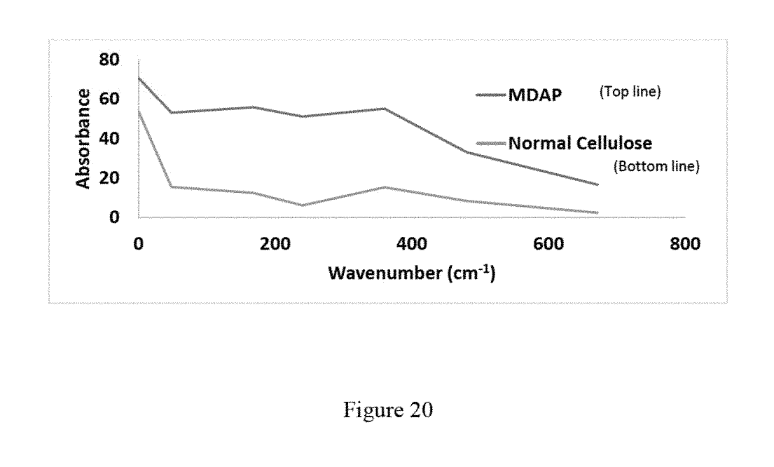

FIG. 21 is a diagram showing a functionalized glucose assay.

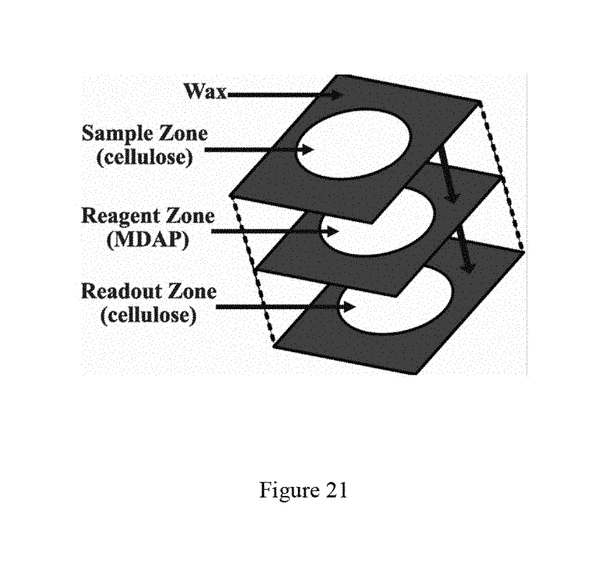

FIG. 22 is a graph (A)) and image (B)) showing a functionalized glucose assay with stability curves.

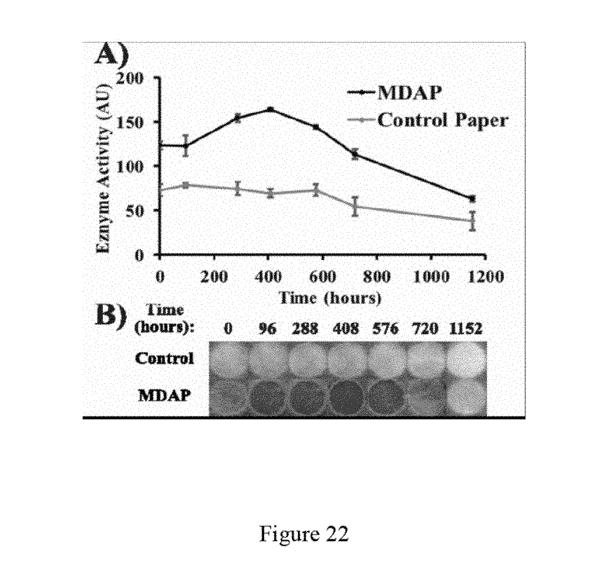

FIG. 23 is a graph showing dye dilution curves.

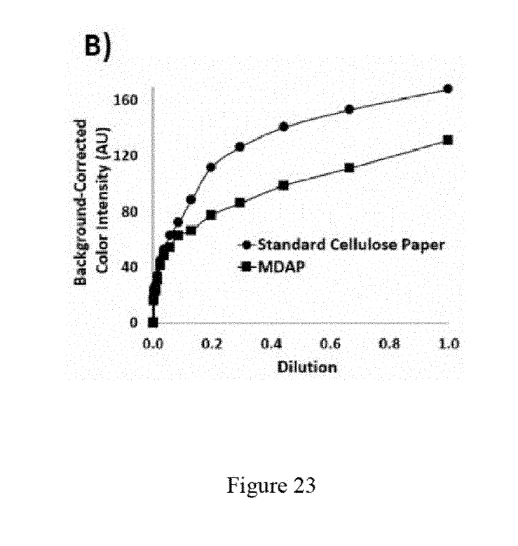

FIG. 24 is a graph showing the percent oxidation of MDAP over time.

FIG. 25 is a graph showing percent degradation of MDAP as a function of the percent oxidation

FIG. 26 is a graphic showing oxidation of glucose subunits.

FIG. 27 is a graphic showing a MDAP comb with wax printing (black) creating a hydrophobic barrier around sample wells (white).

FIG. 28 is a graph showing DNA concentration on control, MDAP or paper. Shown is mean DNA Recovery Concentration (ng/ul; +/− standard error) of Control (n=56), MDAP (n=56), and Paper (n=56).

FIG. 29 is a graph of DNA concentration over time. The relationship between Week and DNA Recovery Concentration is shown as ng/ul. Data points represent the mean of 5 replicates.

### DESCRIPTION

The present methods are useful in producing a paper support for fluid or cells, wherein the support or a portion of the support is contacted with a composition comprising sodium periodate for a period of time until the support surface area is decreased compared to a support not contacted with sodium periodate. A support for fluid includes a fluid with any molecule in solution such as growth factors, enzymes, proteins, nucleic acids, etc.

In an embodiment a portion of the support is contacted with the composition comprising sodium periodate and reduced in size. Another embodiment provides the entire support may be contacted with the composition and the entire support reduced in size. In both instances, as discussed further herein, the contact with the composition comprising sodium periodate reduces surface area and produces dialdehyde paper. This reduced area portion or the entire scaffold having reduced area is degradable.

Where a portion of the support is reduced in size by contact with sodium periodate compositions, the portion in one embodiment may be used to culture cells or hold fluids or the like. The cells or other material may be physically manipulated, and in additional embodiments, the portion which holds or held the cells can be degraded with the remainder of the support remaining. In other words, the cells or other material can be moved via the wax pattern without disrupting the cell layout/distribution.

The paper support will have a particular structure and the miniaturized support will retain the structure. Further the resulting support is malleable and capable of being further shaped as a support for the fluid and/or cells. Hydrophobic barriers such as wax barriers applied to the support are found to function as such barriers even after having been shrunk by the process. Hydrophobic barriers and hydrophilic channels may be produced in the miniaturized form. The process produces a miniaturized/shrunk dialdehyde paper that is retains its original structure yet can be shaped. The support may be formed into a 2D or 3D structure, in an embodiment. A surprisingly high number of hydrophilic channels can be produced with the process, and in one example, 14 parallel hydrophilic channels can be produced. The method may be combined with processes of applying wax on more than one side of the support and subjecting the wax to heat, producing a support that has smaller channels and barriers than can be achieved using the present method and the wax application method separately. What is more, the support is degradable when exposed to an aqueous solution. Thus, a support in one example can be produced by the present methods, cells applied to the support and subsequently the support degraded, such that the cells remain having the form of the support. By way of example without limitation, tissue engineered blood vessels can be produced using such a method. Further, proteins and/or nucleic acid molecules used with the support subjected to the process retain their function and in instances such stability is increased. Proteins, by way of example without limitation, can include proteins that are used in an assay, or are part of the sample to be tested. They include antibodies, enzymes, hormones, proteins that provide structure and support for cells such as actin, or that carry molecules such as ferritin. A nucleic acid molecule (which may also be referred to as a polynucleotide) can be an RNA molecule as well as DNA molecule, and can be a molecule that encodes for a polypeptide or protein, but also may refer to nucleic acid molecules that do not constitute an entire gene, and which do not necessarily encode a polypeptide or protein. The term DNA molecule generally refers to a strand of DNA or a derivative or mimic thereof, comprising at least one nucleotide base, such as, for example, a naturally occurring purine or pyrimidine base found in DNA (e.g., adenine “A”, guanine “G” (or inosine “I), thymine “T” (or uracil “U”), and cytosine “C”). A person of skill in the art recognizes this will include synthetic nucleotide analogs. The term encompasses DNA molecules that are “oligonucleotides” and “polynucleotides”. These definitions generally refer to a double-stranded molecule or at least one single-stranded molecule that comprises one or more complementary strand(s) and “complement(s)” of a particular sequence comprising a strand of the molecule. Stability of nucleic acid molecules and proteins is increased in an embodiment. By way of example without limitation, enzymes used with the scaffolds or nucleic acid molecules such as RNA or DNA placed on the scaffolds in the area that has been reduced in size due to contact with the sodium periodate are stabilized, and in certain embodiments stability is increased.

The process can be tunable, that is, the shrinking process timing controlled by changing concentration of periodate or reaction time to produce a support having a predetermined amount of decrease in surface area and/or percent of oxidation.

Described here are processes for producing a microfluidic paper device or support (sometimes called a chip or microPAD) that is reduced in surface area as a result of exposure to sodium periodate for a sufficient time to cause the surface area of the device to shrink. Also provided here is a biomaterial and scaffold that is inexpensive and simple to fabricate, that can be patterned into distinct zones and that can be shaped into 3D structures.

In an embodiment, the process comprises contacting the device with a composition comprising sodium periodate for a period of time until the device surface area is decreased compared to a device not having been exposed to the sodium periodate.

An embodiment provides the reduction in size is correlated with the percentage of oxidation of the cellulose fibrils. The present of oxidation refers to the percent of glucose sub units that react with periodate. In a preferred embodiment the percent of oxidation is at least 10%, and in further embodiments is 10% to 100%. Oxidation in one example may be measured by a titration method referred to as the Cannizzaro method. Oxidation degree of paper samples was determined in triplicate via the Cannizzaro method (Pommerening et al. (1992) Carbohydr. Res. 233, 219-223. 0.05 g of pDAC sample was weighed and reacted with 10 ml of 0.05M sodium hydroxide for 25 minutes at 70 Celsius with agitation. After allowing to cool to room temperature, 7 ml of 0.1M HCl was added to each sample. Samples were titrated back to a neutral pH using 0.01M NaOH. In order to determine the percentage of glucose monomers converted to dialdehydes, the following equation was used:

OD={[(V1−V2)*N*162]/M}*100

Where V1 is the amount of titrant used, in liters, to bring the solution to a neutral pH, V2 is the amount of titrant used, in liters, to balance a solution containing no oxidized paper sample, N is the molarity of titrant used, and m is the original mass of the oxidized paper sample, in grams.

In a further embodiment, the composition comprising sodium periodate may be aqueous sodium periodate. The composition in an embodiment may comprise at least 0.1M sodium periodate. Further embodiments provide for 0.2, 0.3, 0.4, 0.5, 0.6 0.7, 0.8, 0.9, a saturated solution of 1M, or more, and amounts in-between. The inventors have found that when the composition comprising sodium periodate is heated, increased concentrations of sodium periodate may be used. In an embodiment the composition is maintained at a temperature of 20° C. to 95° C. Embodiments provide more than 1M sodium periodate, and in an example 2M sodium periodate and 2.5M sodium periodate may be used. By way of example, 2.5M sodium periodate may be used when the composition is maintained at a temperature of 95° C. Such higher concentrations lead to faster reactions compared to methods using lower temperatures and using temperatures below 20° C., below 30° C., 35° C., 40° C., 50° C. and below 55° C. In embodiments the reactions proceed at a rate of less than one hour.

Additional embodiments provide the device or portion thereof is contacted with the composition at a volume of up to 0.4 mL sodium periodate per cm² of the device, and in further embodiments at a volume of at least 0.3 mL per cm² of the device. Additional embodiments provide the composition is contacted with the device for at least six hours, in further embodiments for six to 96 hours, and in a preferred embodiment for at least 48 hours and a still further preferred embodiment for 48 hours.

In one example, it is shown that paper patterned via wax printing can be miniaturized by treating it with periodate to produce higher-resolution microPADs. The preferred miniaturization parameters were determined by immersing microPADs in various concentrations of aqueous sodium periodate (NaIO4) for varying lengths of time. This treatment miniaturized microPADs by up to 80% in surface area, depending on the concentration of periodate and length of the reaction time. For example, by immersing microPADs in 0.5-M NaIO4 for 48 hours, devices were miniaturized by 78% in surface area, and this treatment allowed for the fabrication of functional channels with widths as small as 301 μm and hydrophobic barriers with widths as small as 387 μm. The miniaturized devices were shown to be compatible with redox-based colorimetric assays and enzymatic reactions.

The device will have in an embodiment a surface area reduced by up to 75%, and in further embodiments by at least 75%, 76%, 77%, 78%, 79%, 80% or more, or amounts in-between. Embodiments provide the device has at least one hydrophobic barrier and at least one hydrophilic channel. The device in certain embodiments will have at least one barrier having a width decreased by at least 30%. Still additional embodiments provide the at least one channel has a width decreased by at least 49%. Embodiments provide width decrease of barriers and channels of at least 10%, 15%, 20%, 25%, 30%, 35%, 40%, 45%, 50%, 55%, 60%, 65%, 75%, 80%, 85%, 90%, or more and amounts in-between. Further embodiments provide for a decrease in width of both the at least one hydrophobic barrier and hydrophilic channel.

The device is improved by the process by having the ability to increase channel density as a result of reduction in surface area, having decreased average wicking velocity, decreased volume of fluid to fill the at least one channel and resulting increased assay sensitivity. Embodiments provide the channel density increase, decrease in average wicking velocity and decrease in volume fluid to fill can be 2 times, 3 times, 4 times, 5 times, 6 times, 7 times, 8 times, 9 times, 10 times, 11 time, 12, times, 13 times, 14 times, 15 times, 16 times, 17 times, 18 times, 19 times, 20 times, 100 times, 200 times, 300 times or more change from a device not having reduced surface area, and any amounts in-between. The percentage of change can be up to 10%, 15%, 20%, 25%, 30%, 35%, 40%, 45%, 50%, 55%, 60%, 65%, 75%, 80%, 85%, 90%, or more or amounts in-between.

As used herein, the term “microfluidic,” or the term “microscale” when used to describe a fluidic element, such as a passage, channel, chamber or conduit, generally refers to one or more fluid passages, channels, chambers or conduits which have at least one internal cross-sectional dimension, e.g., depth or width, of between about 0.1 m and 1 mm or a volume of a chamber less than 1 microliter (μl). Typically, very small volumes of liquids, from microliters, nanoliters, picoliters to femtoliters are used with the device. The chip in one embodiment can hold fluid. In an embodiment, the chip comprises hydrophilic and hydrophobic channels in which fluids may move, and which can move horizontally or vertically.

In general, microfluidic systems include a microfluidic device, or chip, that has networks of integrated channels in which materials are transported, mixed, separated, and detected. The channels in devices available are microns wide or narrower, and liquids are commonly in the amount of microliters, nanoliters or picoliters. Microfluidic systems may also contain components that provide fluid driving forces to the chip and that detect signals emanating from the chip. Paper-based microfluidic devices typically have channels that are 2 mm wide or narrower, with the height of the channel defined by the thickness of the paper and typically on the order of 200 microns.

The paper contains microfluidic channels in its interior and may include wells on its exterior that provide access to the microfluidic channels. An example of paper microfluidic chips is that described at U.S. Pat. No. 9,791,434, incorporated herein by reference in its entirety. In that example, there is provided a stem and plurality of branches or channels. In embodiments such channels can be hydrophilic. At least one barrier may also be provided that is hydrophobic. These barriers may be placed to help contain fluid within the device and/or channels.

The chip in an embodiment will have a plurality of channels. The precise form of the channels varies depending upon the application and the precise number or layout of channels can be any convenient form and there can be any number that is suitable for the application. For example, it may have at least one such channel, preferably more than one channel, and can have up to 10, 20, 100, 1000 chambers or more or any amount in-between. Any chambers provided may be connected to other channels and the chambers and/or channels connected to each other.

Other features may be optionally included with the chip, and it may for example use passive fluid control techniques or micropumps or micro valves or other enhancements.

Optionally such chips may have components for detection and separation of analytes and one skilled in the art will select components and methods to include useful for the particular application. They may, for example, use electrochemical, luminescence, whether chemical or electrical, colorimetric detection or any other useful method.

The present processes and compositions are not limited to a particular means of producing the paper microfluidic chip that will be subject to the methods described. One example of producing paper microfluidic devices uses a chemical vapor deposition reactor chamber at US Application 20160339428. A wide range of methods to produce such devices is and will be available including, for example, wax printing (typically involving printing and heating wax patterns onto paper), inkjet printing, photolithography (using a mask/patterned screen and light exposure to create patterns), flexographic printing (employing a flexible relief plate with a raised image of the pattern and ink), plasma treatment (where paper is hydrophobized and then treated with plasma and a mask), laser treatment, wet etching (where hydrophilic filter paper is patterned using a mask and an etching agent), screen-printing (using a patterned screen and a chemical such as polystyrene) and wax screening (where wax is pressed though a screen onto paper and melted).

The use of microfluidic technology is intended for use in a wide variety of applications including use for a number of analytical chemical and biochemical operations. This technology allows one to perform chemical and biochemical reactions, macromolecular separations, and the like, that range from the simple to the relatively complex, in easily automated, high-throughput, low-volume systems. Further information about microfluidic devices and systems is presented in U.S. Pat. No. 6,534,013. This reference and all references cited herein are incorporated herein by reference in their entirety. For example, they may be used in biological and industrial applications. Devices that operate on such miniaturized scale are often called “lab-on-a-chip” in their capacity to bring to the field analysis that typically would occur in a laboratory. Examples, without being limiting, are in DNA amplification, electrophoresis, chromatography, staining, fluorescence cytometry, protein analysis, polymerase chain reaction, blood or other animal fluids analysis, or Fluorescence In Situ Hybridization (see, e.g., U.S. Pat. No. 9,364,830 for a discussion of FISH). They are particularly useful in medical and health care applications, such as diagnosis, delivery of drugs or other compounds, and especially for point of care applications where delivered at the location of the animal or patient. Sample analytes include, without limitation, urea, creatinine, creatine, glucose, lactate, ethanol, uric acid, cholesterol, pyruvate, creatinine, β-hydroxybutyrate, alanine amino transferase, aspartate aminotransferase, alkaline phosphatase, and acetylcholinesterase. Still further examples of uses are in detecting the presence of viruses, bacteria and other microorganisms which may be pathogen (see, e.g. US Patent Application 2015030902). In another example, chemical compounds that may be toxins can be detected. A further example is use in detection of bodily fluids for forensic serology (see e.g., US Application 20170067881). Advances continue in producing micro organs on chips, which have the purpose of reproducing key functions of living organs. Examples of industrial uses include micro-thermal technologies, and the manufacture of micro structures in applications such as advanced cooling systems, medical sensing systems, tunable microlens arrays (see, e.g., Miccio et a (2008) Optics Express, Vol. 16, No. 11), and as in the microfluidic device origin, with inkjet printheads.

Here is described a new approach for preparing microPADs, which in an embodiment provides for higher-resolution features by miniaturizing lower-resolution wax patterns in paper.

The concept of shrinking materials in order to fabricate small devices and structures has been explored most famously by the Khine group.¹³⁻¹⁵ They used Shrinky-Dinks and other thermoplastic shrink films, which shrink up to 95% in surface area when exposed to heat, to fabricate plastic or polymer-based microfluidic devices as well as other microstructures and metallic nanostructures.¹⁵ Hydrogels, which can shrink upon drying or in response to changes in environmental conditions like pH or temperature, have also been used to fabricate small structures and patterns.^{16,17} The advantage of using shrinkable materials for fabrication of small structures is that it is relatively easy to pattern or fabricate larger, lower-resolution structures, and these can then be converted into smaller, higher-resolution structures upon shrinking without the need for sophisticated microfabrication equipment.

Paper is not commonly thought of as a material that shrinks-even though we probably all have some experience with shrinking cotton cloth, another cellulose-based material, when doing laundry.^{18,19} However, after an extensive review of the literature, we identified two methods for shrinking paper. The first method involved multiple cycles of soaking paper in liquid ammonia followed by drying.²⁰ This approach was used to shrink a dollar bill by ˜55% in surface area—the bill shrank anisotropically in plane by ˜38% in length and ˜28% in width.²⁰ We did not investigate this method due to the risks of working with liquid ammonia. The second method, and the one that we explored for miniaturizing microPADs, involved soaking paper in aqueous solutions of periodate.²¹

Periodate oxidation of cellulose via the Malaprade reaction has been investigated extensively in the context of producing derivatives of cellulose.²²⁻²⁹ Periodate oxidation of paper has also been investigated previously as a method for covalently linking molecules to paper.³⁰⁻³³ When paper was soaked in dilute solutions of periodate (<0.1 M) or for short reaction times (<1 h), we found that the dimensions of the paper were not affected by the chemical treatment. But, if a concentrated solution of periodate was used over longer periods of time, then the paper shrunk. The earliest reference to the shrinkage of paper upon exposure to periodate that we could find states that filter paper could be shrunk to 25% of its original surface area (i.e., by 75% in surface area) by exposing it to 0.271-M periodic acid in water for 37 days.²¹ The shrinkage of paper was later attributed to a reorganization of the oxidized cellulose chains into non-linear conformations that led to buckling and ultimately to shrinking of the oxidized cellulose fibers.²⁷ Periodate oxidation has also been used to shrink cotton cloth and cotton string,³⁴ but has not, to our knowledge, been investigated previously for the purposes of device fabrication. We now demonstrate that microPADs prepared via wax printing can be shrunk using periodate oxidation to fabricate miniaturized devices with higher-resolution patterns.

Wax printing is one of the most common techniques for patterning paper to fabricate microPADs.³⁵⁻³⁷ In this approach, wax is printed onto paper using a solid-ink printer, and then the paper is heated to reflow the wax so that it seeps into the paper and creates a hydrophobic barrier.³⁵ One limitation of wax printing is the relatively low resolution of the technique, a result of the wax boundaries spreading laterally as well as vertically during the heating step.¹² There is one example of using wax printing to produce high-resolution, sub-millimeter patterns, which was achieved by Tenda et al. by printing wax on both sides of the paper followed by a brief heating step using a thermal laminator.¹² Two other techniques for producing sub-millimeter-scale patterns in paper rely on photolithography and laser cutting, respectively.^{38,39} To fabricate our high-resolution microPADs, we first optimized the chemical reaction required for miniaturization, we then characterized the miniaturized devices, and, finally, we demonstrated some of the potential advantages and applications of this new type of paper-based device.

What is more, the present processes provide for preparation of the device as a scaffold, and in an embodiment, is a degradable scaffold useable in a number of applications such as cell culture and in tissue engineering. The scaffold produced here is biocompatible, degradable under physiological conditions and has the appropriate structural and mechanical properties to facilitate the growth of cells including aggregates of cells such as tissues. [B 12, 14, 17] A scaffold here is used to provide a framework or structural element that holds tissues or cells together. In certain embodiments, a scaffold can mimic the extracellular matrix of biological tissue.

There are many means for forming paper into a scaffold, and the processes and devices here are not limited by the means of forming the scaffold structure. For example, Derda et al. describes the preparation of scaffolds by stacking layers of chromatography paper impregnated with cell suspensions in an extracellular matrix hydrogel. Derda et al. (2009) “Paper-supported 3-D cell culture for tissue-based bioassays” PNAS, Vol. 104 No. 44, pp. 18457-18462. A hydrogel precursor with suspended cells is added to the paper support and gelled in place. Though chromatography paper that is 200 μm thick was used, the authors note paper of other types can be used, including, by way of example without limitation, such paper as lens paper with a thickness of 30 μm to blotting paper with a thickness of 1,500 μm. The stacked layers may be destacked for analysis. Park et al. describes initiated chemical vapor deposition (iCVD) processes for coating a water repellant and cell adhesive polymer film to make paper scaffolds. Park et al. (2014) “Paper-based bioactive scaffolds for stem cell-mediated bone tissue engineering” Biomaterials 35:9811-9823. An origami-based approach is described by Kim et al. which can be enhanced through computer-aided design. Kim et al. (2015) “Hydrogel-laden paper scaffold system for origami-based tissue engineering” PNAS Vol. 112, No. 50 pp. 15426-15431. A hydrogel layer with chondrocytes was deposited with iCVD process and formed into the shape of rabbit trachea. Three-D printing is yet another example of preparation of a scaffold. Gross et al. (2014) “Evaluation of 3D printing and its potential impact on biotechnology and the chemical sciences” Anal. Chem. 86(7):3240-3253. Many variations on methods of producing a scaffold are and will become available to a person skilled in the art.

A person of skill in the art also appreciates there are a myriad of uses of such scaffolds. By way of example without limitation, such scaffold may be used in tissue engineering (see Kim et al, and Park et al, supra); for bioassays (see Derda et al., supra); as disease models which can be used to create various microenvironments; in the study of cancer cells and tumors; in drug screening to monitor cell response; and in cell cryopreservation (See Ng et al., (2017) “Paper-based cell culture platform and its emerging biomedical applications” Materials Today Vol. 20 No. 1 pp. 32-44).

Here the material used for a microfluidic chip described above or scaffold is paper. When referring to paper here is meant a bundle of cellulose fiber and which in an embodiment may be a sheet of paper. Paper for use in biomedical applications is described by Ng et al. with various examples provided of cellulose fibers held by hydrogen bonds. Ng. et al. supra. Examples include WHATMAN® filter paper, Janus paper, weighing paper, print paper and even KIMBERLY-CLARK® KIMWIPES®.

The devices may be made of one or more sheets of paper. Paper has been used extensively in the research laboratory,[B1, 2, 18] and cellulose-based paper has been explored recently as a scaffold for 3D cell culture and tissue engineering.[B3, 19-24] Some of the main advantages of working with paper are that it is a low-cost material, it is biocompatible, it can be patterned easily with waxes and other reagents using commercial printers, it can be modified chemically, it can be stacked, folded and shaped into more complex 3D structures (e.g., origami), and it is made of a network of cellulose fibers that inherently present the appropriate pores for culturing cells.[B3, 1, 20, 21] Paper is also available commercially in a wide variety of forms with different thicknesses and pore-sizes, so different papers could be selected for different applications.[B2] While paper has many appealing characteristics, an important limitation of paper as a scaffold for cell culture and tissue engineering is that it does not degrade under physiological conditions. Thus, a paper scaffold could interfere with potential downstream applications of cells or tissues cultured in paper. For example, in the context of cell culture, the paper matrix may interfere with techniques for imaging the cells. In the context of tissue engineering, the paper matrix may not be suitable for implantation. By modifying paper chemically, we describe here a process for retaining all the positive characteristics of paper as a scaffold while making it degradable.

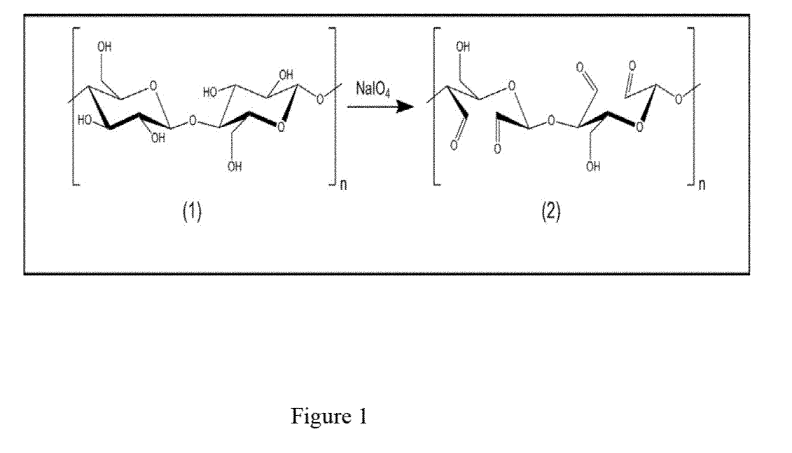

FIG. 1 shows oxidation of cellulose (1) with sodium periodate to generate 2,3-dialdehyde cellulose (2). The production of 2,3-dialdehyde cellulose (DAC) via the Malaprade reaction as shown has been investigated for producing derivatives of cellulose. (B25-35) DAC is interesting in the context of this proposal because, unlike cellulose, it degrades in water and was even shown to degrade into small molecules like glycolic acid and 2,4-dihydroxybutyric acid after being implanted in rats.[35-38] Furthermore, biodegradable scaffolds prepared from DAC have been demonstrated successfully for cell culture.[39, 40] These scaffolds were prepared from methylcellulose, a soluble cellulose derivative, that was cast into thin films and then oxidized with periodate,[39] or from bacterial cellulose that was biosynthesized by Acetobacter xylinum and then oxidized with periodate.[40]

Here we describe miniaturized dialdehyde paper (MDAP) as a promising new biomaterial for manufacturing scaffolds for cell and tissue culture, and, to our knowledge, no one has explored this possibility previously. The potential advantages of MDAP are that it is easy to prepare from off-the-shelf materials, it possesses all the same advantages as conventional paper (i.e., it can be patterned, it can be shaped into 2D and 3D structures, and it is porous) and, like DAC, it should be biocompatible and degradable in water. Any solution that degrades could be employed and other examples include 1× phosphate buffered saline solution (1×PBS) and Dulbelco's Modified Eagles Medium w/10% Fetal bovine serum (Complete DMEM+10% FBS). These last two were found to lead to a significantly more rapid degradation of the MDAP. An added advantage of MDAP, that to our knowledge has not been explored previously, is its tunable miniaturization upon oxidation that enables the fabrication of small structures, which may not be accessible via other fabrication methods.

The following is provided by way of exemplification and is not intended to limit the scope of the invention.

### EXAMPLES

### Example 1

### Standard MicroPAD Fabrication

Standard microPADs were fabricated via wax printing.³⁵ The patterns for the devices were designed in Adobe Illustrator (CS6) and printed onto Whatman No. 1 CHR chromatography paper using a solid ink printer (Xerox Phaser 8650). After printing, the sheets of paper were heated for 2 minutes in a convection oven (MTI corporation, Compact Forced Air Convection Oven) set to 195° C. The devices were then cooled to room temperature, cut out with scissors, and stored under ambient conditions until used.

### Optimization of MicroPAD Miniaturization

Solutions of sodium periodate (NaIO4) with concentrations of 0.1, 0.2, 0.3, 0.4, 0.5 and 1.0 M were prepared in deionized (DI) water. The solubility of NaIO4 in DI water at room temperature was found to be approximately 0.5 M, and the 1.0-M solution that was prepared was a saturated solution containing solid NaIO4. Standard microPADs with dimensions of 4.50 cm×4.50 cm were immersed in 25 mL of each periodate solution at room temperature in a covered glass Petri dish.

The Petri dishes were shielded from ambient light during the reaction. Devices were removed from the periodate solution after a given reaction time ranging from 6 hours to 96 hours. The devices were then washed by placing them in a bath of DI water for 15 minutes with rocking. After washing, the devices were dried for 1 h in a slab gel dryer (Bio-Rad Model 443) at 60° C. and 300 torr. The miniaturized devices were measured with a ruler.

The effect of the wax patterns on the miniaturization process was studied by miniaturizing microPADs with a full wax background, microPADs with wax-outlined channels, and paper with no wax patterns in 0.5-M NaIO4 for various time intervals up to 96 hours. The devices were washed, dried and measured as described previously.

The minimum volume of NaIO4 solution required for miniaturization was determined by miniaturizing standard microPADs in varying amounts (2-10 mL in 1 mL increments) of 0.5 M NaIO4 for 48 hours.

Fabrication of Miniaturized microPADs

To fabricate miniaturized microPADs, devices patterned via wax printing were soaked in 0.5-M NaIO4 for 48 hours. The volume of NaIO4 solution used to miniaturize a given microPAD was determined from the initial surface area of the device (in cm²). At a minimum, 0.4 mL of NaIO4 solution per cm² of device was used. After miniaturization, the devices were washed and dried as described previously. A more detailed description of the procedure for preparing miniaturized microPADs is provided in the supplementary information.

### Characterization of Miniaturized MicroPADs

The minimum functional hydrophobic barrier width and minimum functional hydrophilic channel width were determined for both standard and miniaturized microPADs. A functional hydrophobic barrier was defined as a barrier that prevented aqueous colored dye from wicking across it for at least 30 minutes, and a functional hydrophilic channel was defined as a 5-mm-long channel that could wick aqueous colored dye from a fluid reservoir to a test zone.¹² To determine the minimum functional hydrophobic barrier width, a series of barriers with varying widths (designed in Adobe Illustrator with dimensions in the range of 100-800 m) were fabricated and then tested by adding 10 μL of an aqueous colored dye solution (either 1-mM Erioglaucine blue dye or 5-mM Allura Red dye in DI water) to one side of the barrier, while looking for evidence of passage of fluid or leakage on the other side of the barrier after 30 minutes. The final barrier widths were measured using a dissecting microscope (400× magnification) equipped with a digital camera and a stage micrometer. To determine the minimum functional hydrophilic channel width, a series of channels with varying widths (designed in Adobe Illustrator with dimensions in the range of 500-1200 m) were fabricated and then tested by adding 20 μL of aqueous dye to a fluid reservoir on one side of the channel and monitoring passage of the fluid to a test zone on the opposite side of the channel. Final channel widths were also measured using a dissecting microscope.

The surface and cross-section of pieces of chromatography paper and miniaturized chromatography paper (with no wax patterns) were imaged with a scanning electron microscope (SEM, FEI Quanta 200). The height (thickness) of each piece of paper was determined from the SEM images.

The average wicking velocity was determined for both standard and miniaturized microPADs by adding 15 μL of aqueous dye to a sample zone leading into a channel (1.5 mm in width, 10 mm in length) and measuring the time required for the fluid to wick across the channel. The average wicking velocity was calculated by dividing the length of the channel by the wicking time.

The minimum volume of fluid required for wicking across a 5-mm-long channel for both standard and miniaturized microPADs was measured. The determined minimum functional hydrophilic channel widths for each type of device were used (standard device: 0.6 mm, miniaturized device: 0.3 mm). A range of fluid volumes (0.5-10 μL in 0.5 μL increments) were added to the channels, and the minimum amount of fluid required to fill the channels was recorded.

### Confirmation of Miniaturized MicroPAD Functionality

Glucose Assay:

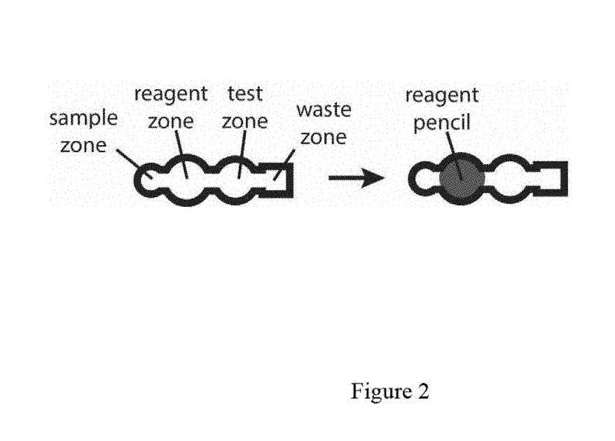

Miniaturized microPAD functionality was confirmed by performing a glucose assay on a miniaturized device with a sample zone, a reagent zone, a test zone and a waste zone all connected in series by a straight channel (FIG. 2).⁴⁰ The reagents for the assay were deposited onto the reagent zone using a reagent pencil, which was fabricated as described previously by pressing a mixture of 66.6% w/w polyethylene glycol (Mn 2000 g/mol), 22.2% w/w graphite powder, 0.75% w/w glucose oxidase (GOx, 266 U/mg), 0.52% w/w horseradish peroxidase (HRP, 293 U/mg), and 10.0% w/w 2,2′-azino-bis(3-ethylbenzothiazoline-6-sulphonic acid) (ABTS) into the shape of a cylindrical pellet with a diameter of 3.2 mm using a manual pellet press (Parr Instrument Company).^{40,41} Glucose solutions (3.5 μL) prepared in 1×PBS with concentrations of 0, 0.3, 0.6, 0.9 and 1.2 mM were applied to the sample zone of the device and a colorimetric readout was generated in the test zone. The intensity of the color produced in the test zones was measured via digital image colorimetry (DIC),⁴² where the mean color intensity in the red channel of the test zones was measured using a smartphone (Samsung Galaxy Note 4) and the Color Grab app.⁹

Enzyme Viability:

Solutions of horseradish peroxidase (HRP, 0.6-10.5 U/mL in 1×PBS, 2 μL) were added to circular test zones (5.5 mm in diameter) on miniaturized microPADs.

Immediately after drying the HRP solutions on the devices under ambient conditions, 3 μL of tetramethylbenzidine liquid substrate (TMB, Sigma Aldrich, T4444) was added to each test zone, and the reaction was allowed to proceed for 20 minutes. Sulfuric acid solution (H₂SO₄, 1 M in DI water, 2 μL) was added to each test zone to quench the reaction, and the test zones were dried under ambient conditions. The mean intensity of each zone was measured as described previously.

### Results and Discussion

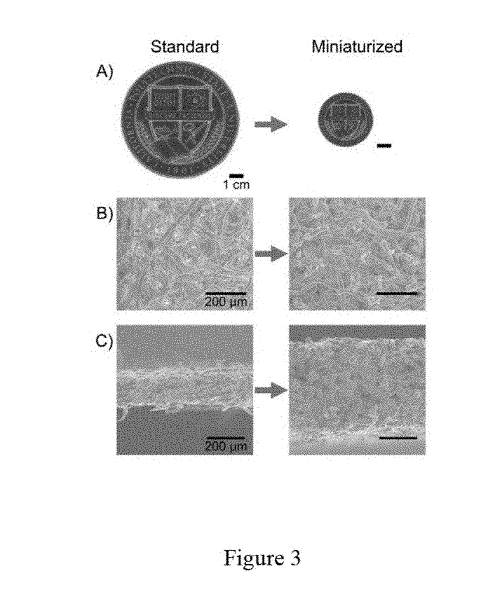

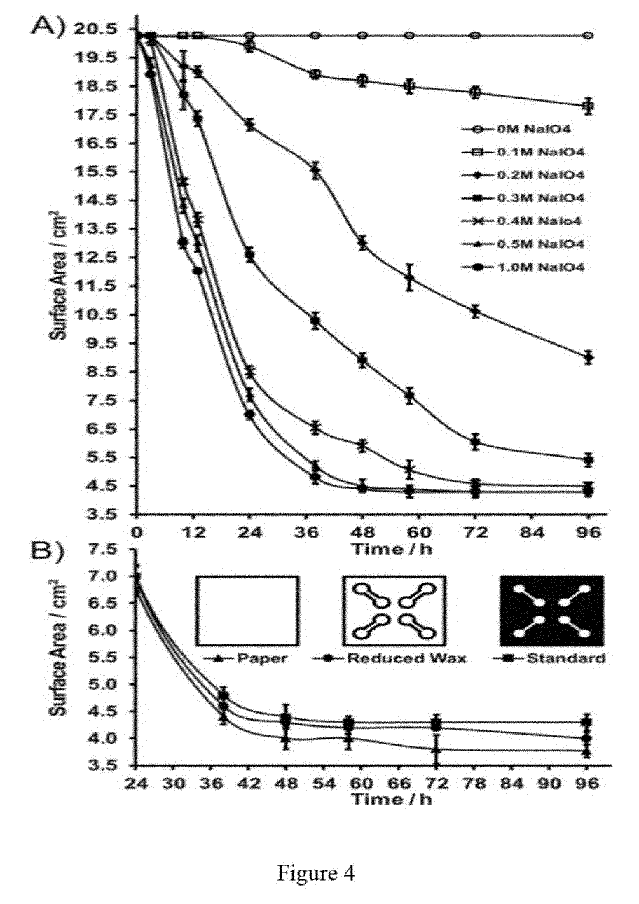

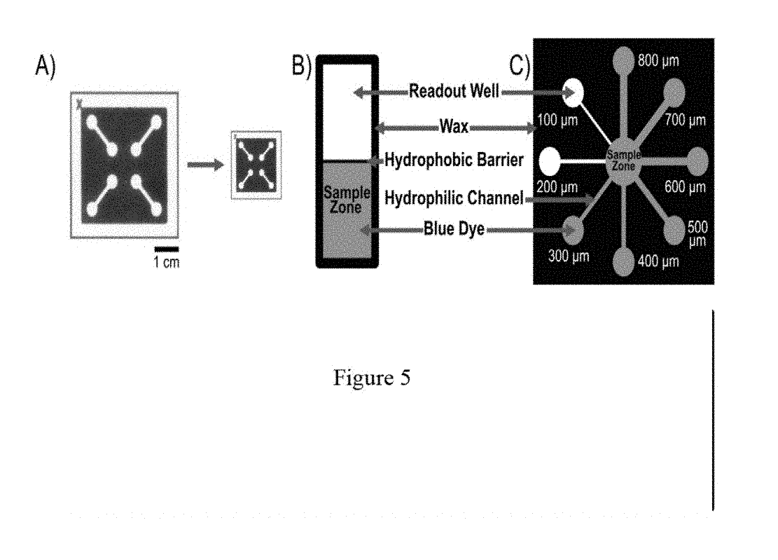

Miniaturized microPADs were fabricated by immersing standard, wax-printed devices in aqueous solutions of periodate for varying lengths of time (FIG. 4A). Upon reacting with periodate, the devices shrunk in the plane of the paper, while the height (thickness) of the paper increased, which is analogous to what is observed when shrinking thermoplastic shrink films.¹³ The wax barriers shrunk proportionally with the paper resulting in high-fidelity, miniaturized reproductions of the original standard devices (FIG. 3A & FIG. 5A). SEM images comparing a piece of untreated chromatography paper to a piece of miniaturized chromatography paper show that the paper fibers appear to swell and pack more densely during the miniaturization process (FIG. 3B, C). The images also suggest that the size of the pores in miniaturized paper are smaller compared to the original paper.

The degree of miniaturization of microPADs can be controlled by tuning both the concentration of periodate and the reaction time (FIG. 4A). For example, when 0.1-M periodate was used, the devices shrunk more slowly, and the reaction needed at least 24 hours before any change in size was observed. When 0.5-M periodate was used, some miniaturization was observed after only 6 hours, most of the miniaturization took place within 48 hours, and no additional miniaturization was observed after 72 hours. The amount of wax patterning on microPADs had only a small impact on the degree of miniaturization (FIG. 4B). After 96 hours in a 0.5-M periodate solution, full-wax devices shrunk 78.6% in surface area, wax-outlined devices shrunk 79.8% in surface area, and paper without wax patterns shrunk 80.9% in surface area. One possible explanation for these results is that the wax patterns protected some of the cellulose molecules from reacting completely with the periodate, which may have slightly limited the degree of miniaturization in the case of the full-wax devices.

Since our goal with this project was to establish a method for miniaturizing microPADs, we selected 0.5-M periodate and 48 hours of reaction time for the optimized miniaturization procedure. Higher concentrations of periodate cannot be achieved due to the solubility of NaIO4 in water at room temperature, and a saturated solution of periodate (e.g., 1.0 M) did not shrink the devices any further or faster than the 0.5-M solution. Longer reaction times than 48 hours did not result in significant additional miniaturization either. Devices that were miniaturized for 72 or 96 hours were only 0.5% smaller than devices miniaturized for 48 hours (FIG. 4).

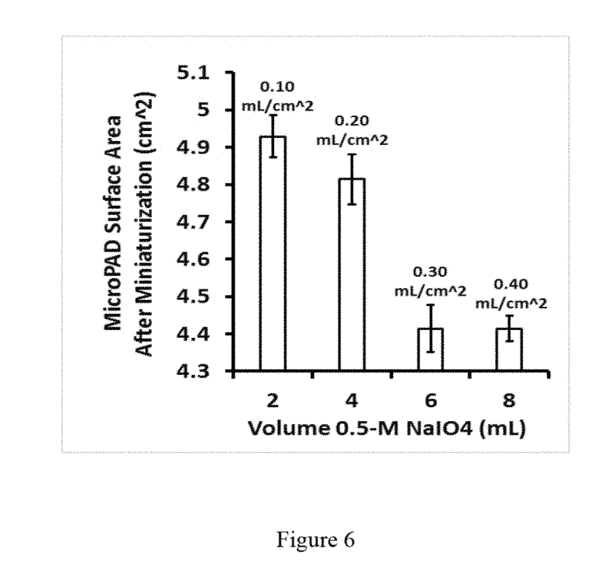

After reacting in 0.5-M periodate for 48 hours, the average reduction in size for a standard microPAD was 78% in surface area, or 53% in linear dimensions (FIG. 4B, 5D). To achieve this level of miniaturization, a minimum of 0.3 mL of 0.5-M periodate solution per cm² of microPAD surface area was required (FIG. 6). These results indicate that a minimum of 0.30 mL 0.5-M NaIO4 per cm2 of microPAD is preferred for complete miniaturization. When lower volumes of solution were used, the devices did not shrink to the same extent. Additional solution, above 0.3 ml/cm², had no effect on the miniaturization process, therefore we recommend using 0.4 ml/cm² of the periodate solution to ensure proper miniaturization.

For miniaturized microPADs, the narrowest functional hydrophobic barrier had an average width of 387±20 m (designed as 220 μm in Adobe Illustrator) (FIG. 5C, D), and the narrowest functional hydrophilic channel had an average width of 301±42 (designed as 1100 μm in Adobe Illustrator) (FIG. 5B, D). For comparison, the narrowest hydrophobic barrier in a standard microPAD had an average width of 555±37 μm (designed as 180 μm Adobe Illustrator), and the narrowest hydrophilic channel had an average width of 585±54 μm (designed as 1100 μm in Adobe Illustrator). The differences represent a 30% reduction in the width of the smallest hydrophobic barriers and a 49% reduction in the width of the smallest hydrophilic channels for miniaturized microPADs compared to standard microPADs. The ability to fabricate microPADs with smaller, higher-resolution features should allow for higher channel density to be incorporated into microPADs. For example, based on the determined minimum hydrophilic channel and hydrophobic barrier widths, a 1-cm-wide microPAD could theoretically accommodate up to 8 parallel hydrophilic channels in the case of a standard microPAD, but could accommodate up to 14 parallel hydrophilic channels in the case of a miniaturized microPAD. Compared to the method published by Tenda et al., which reported a minimum hydrophobic barrier width of 467±33 μm a minimum hydrophilic channel width of 228±33 μm,¹² our method allows for the fabrication of smaller hydrophobic barriers but slightly larger hydrophilic channels. An interesting observation is that the two methods of fabrication are orthogonal and could potentially be combined to fabricate devices with even smaller channels and barriers than could be achieved using either method independently.

The average wicking velocity in miniaturized microPADs was reduced by a factor of ˜2 compared to standard devices (Table 1). Fluid wicked across channels (1.5 mm in width×10 mm in length) in miniaturized devices in 42±3 s, for an average rate of 0.24±0.02 mm/s, while fluid wicked across channels with the same dimensions in standard devices in 21±4 s, for an average rate of 0.48±0.08 mm/s. The decrease in average wicking velocity can likely be attributed to a combination of two factors: a decrease in the effective pore size and an increase in hydrophobicity of the miniaturized paper. When shrinking paper, the cellulose fibers contract and pack more tightly, which, in turn, leads to smaller spaces between the fibers, as was observed by SEM. Smaller pores would be expected to slow down wicking as predicted by the Lucas-Washburn model.⁴³⁻⁴⁶ Periodate oxidation of paper also reduces the number of hydroxyl groups on paper, which would increase the hydrophobicity of the resulting material compared to untreated paper and also contribute to slower wicking. Slower wicking will not necessarily impact the performance of miniaturized devices since these devices would typically be smaller than standard microPADs, so the fluid would be wicking over shorter distances. Slower wicking rates could also allow for increased assay sensitivity by increasing reaction time within channels and test zones. Future microPADs could also potentially incorporate both standard and miniaturized paper in multi-layered devices to harness the advantages of both materials.

A final important characteristic for microPADs is the volume of fluid required to fill the device. We found that a miniaturized microPAD required 2 μL to fill a 5-mm-long channel, while a standard microPAD required 8 μL to fill a channel of the same length (FIG. 4D). The reduction in volume of fluid can be attributed to two effects. First, smaller channels can be fabricated in miniaturized microPADs, therefore these channels will require less fluid. In our experiment, the width of the channel in the miniaturized microPAD was 0.3 mm and the width of the channel in the standard microPAD was 0.6 mm. Second, because the fibers in the miniaturized devices are packed more tightly, there is less void space in the miniaturized devices that can fill with fluid. In general, lower volume requirements are favorable since they allow for assays to be performed on smaller sample sizes, and these devices may also function with smaller quantities of reagents.

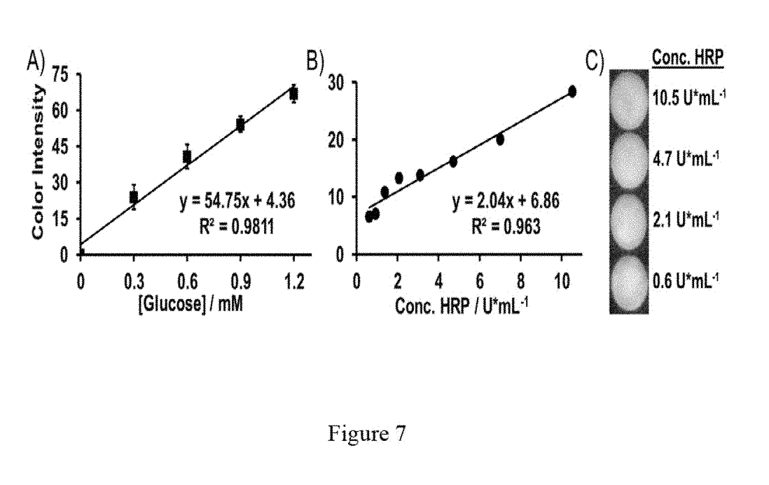

The performance of miniaturized microPADs as platforms for biochemical assays was confirmed by performing a glucose assay (FIG. 7A). The results were quantified via DIC and were used to generate a linear calibration curve with a high R² value (0.98). In addition to analytical performance, the glucose assay confirmed enzyme functionality on miniaturized microPADs as both GOx and HRP activity are required for the assay. Furthermore, since this assay relies on redox chemistry, it demonstrated that the periodate was either completely removed from the miniaturized devices during the wash step or that any residual periodate did not interfere with the assay.

The viability of enzymes on oxidized cellulose fibers was also confirmed by performing a colorimetric assay for HRP on miniaturized devices. After drying the HRP solutions on the devices, a concentration-dependent color intensity was produced upon addition of the substrate for the enzyme (FIG. 6B, C). This result is significant given that several diagnostic assays rely on the activity of enzymes for signal amplification or for direct detection of analytes.⁴⁷

We developed a new method for fabricating microfluidic devices having reduced surface areas. These devices in an embodiment may be higher-resolution microPADs made by shrinking devices such as wax-patterned devices. Miniaturized devices can incorporate higher channel density and can be used as platforms for the same types of biochemical assays that are typically performed on standard microPADs. The miniaturized devices also require smaller volumes of sample per unit surface area of the device.

The method for shrinking microPADs is highly tunable and can be controlled easily by changing the concentration of periodate or the reaction time. This method could also be readily applied and adapted toward the fabrication of other types of devices or structures. We believe that the miniaturization process will allow for the fabrication of smaller point-of-care diagnostics, and we are currently exploring additional applications for miniaturized microPADs.

### Example 2

The following is an example employing methods described herein.
1. Design microPADs using a drawing program (e.g., Adobe Illustrator). When designing the devices, it is important to account for lateral wax diffusion during heating and the subsequent miniaturization. Printed wax barriers will diffuse ˜275 μm in every direction in the plane of the paper upon heating, followed by a ˜53% reduction in linear dimensions upon miniaturization.
2. Print the designs onto chromatography paper (CHR 1) using a solid-ink printer (e.g., Xerox Phaser 8560).
3. Heat the printed pages using a forced air convection oven (or other heat source) set to 195° C. for 2 min.
4. Prepare a 0.5-M solution of sodium periodate in deionized water (Note: periodate is a strong oxidizer and should be handled with care. Please refer to the SDS for sodium periodate for information on proper handling of this reagent). At least 0.4 mL of solution per cm² of microPAD surface area should be prepared.
5. Submerge the devices in the periodate solution in an appropriately-sized container made from glass or plastic. Cover the container and place in a dark area, and allow the reaction to proceed for 48 h.
6. Remove the miniaturized microPADs from the periodate solution and wash them in deionized water for 15 minutes. Placing the water bath on a rocker will promote water flow over the devices. Failure to properly wash any remaining periodate out of the paper will lead to yellowing and increased hydrophobicity of the paper. Dispose of the remaining periodate solution in an appropriate waste container.
7. Place the devices in between two pieces of blotting paper in a gel slab dryer for 1 h (60° C., 300 torr).

### Example 3

The experiment is organized into three main parts: (i) development and characterization of miniaturized dialdehyde paper (MDAP) as a new biomaterial, (ii) evaluation and optimization of MDAP as a scaffold for cell culture and tissue engineering, and (iii) application of MDAP as a scaffold for preparing tissue-engineered blood vessels.

### Development and Characterization of MDAP as a New Biomaterial.

The first phase of the experiment will involve the preparation of MDAP from a variety of different types of paper. We will concurrently characterize the mechanical and structural properties of MDAP and focus on studying the rate of degradation of MDAP in aqueous solutions. We will also investigate approaches for tuning the rate of degradation of MDAP. We will develop methods for patterning and shaping MDAP into 2D and 3D structures.

### Development of Methods for Preparing MDAP.

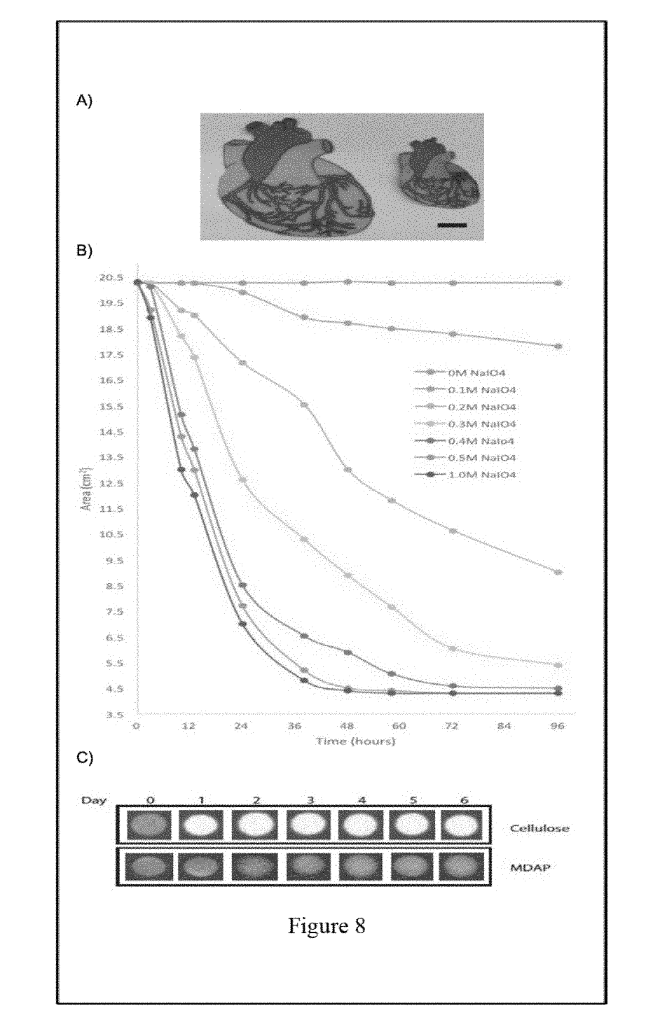

MDAP can be prepared by soaking cellulose-based paper in aqueous solutions of sodium periodate (FIG. 8A).[B30] We have performed experiments using Whatman Grade 1 Chr paper and have found that the progress of the oxidation reaction and the degree of miniaturization of the paper are a function of both time and the initial concentration of periodate (FIG. 8B). We have found that Whatman Grade 1 Chr paper can be miniaturized to ˜25% of its original surface area when exposed to concentrated periodate solutions (>0.3 M) for ˜24-72 h.

This experiment tests different types of paper, different concentrations of periodate, different reaction times, different reaction conditions (e.g., temperature, agitation), different rinsing protocols and different drying conditions. Pieces of paper cut into 4 cm×4 cm squares will be labeled and weighed, and their height (thickness) will be measured using calipers. The papers will then be soaked in a given periodate solution in a glass petri dish for a defined period of time under a defined set of reaction conditions. The reaction will be kept in the dark as periodate is known to degrade upon exposure to UV light.[39] The resulting MDAP will then be rinsed, dried and then the final dimensions (length, width and height) and mass of the MDAP will be determined. The MDAPs will then be further characterized based on degree of oxidation, structural and mechanical properties (see above) and rate of degradation in aqueous solution (see above). All experiments will be performed in triplicate.

The initial set of experiments will be performed using Whatman Grade 1 Chr paper as the starting material. Once a reliable protocol for producing MDAP has been established, we plan to investigate the use of other papers with different characteristics such as thickness and pore size in order to produce MDAPs with varying characteristics (Table 2).[B 42]

TABLE 2
Types of paper that will be evaluated for producing MDAP.

| | | | Wicking |
|---|---|---|---|
| | Thickness | Particle retention* | rate* |
| Paper type | (μm) | (μm) | (mm/30 min) |
| Whatman chromatography | | | |
| paper: | | | |
| Grade 1 Chr | 180 | NA | 130 |
| Grade 3 Chr | 360 | NA | 130 |
| Grade 20 Chr | 170 | NA | 85 |
| Grade 31ET Chr | 500 | NA | 225 |
| Grade 2668 Chr | 900 | NA | 155 |
| Whatman filter paper: | | | |
| Grade 1 | 180 | 11 | NA |
| Grade 4 | 205 | 20 | NA |
| Grade 5 | 200 | 3 | NA |

*Particle retention and wicking rate correlate with the mean pore size in paper. Smaller particle retention and slower wicking rates typically correspond to smaller pore sizes.

We plan to test initial periodate concentrations in the range of 0.1 M to 1.0 M. The solubility of sodium periodate at room temperature is ˜0.5 M, so solutions prepared above this concentration may be saturated depending on the temperature. Reaction times of 3, 6, 12, 24, 36, 48, 60, 72 and 90 h will be tested. The progress of the reaction will be tracked using the dimensions of the MDAP as well as the degree of oxidation of the MDAP. In one set of 16 experiments, MDAP will be prepared at three different temperatures: 4° C., 20° C., and 40° C., in order to determine the effect of temperature on the reaction. The effect of the initial amount of periodate solution (per g of paper) on the preparation of MDAP will be studied. Other potential experiments involve preforming the reaction on a laboratory rocker to continuously stir the periodate solution, and to determine the effect of replacing the periodate solution with fresh solution at various time points.

Once the reaction is complete, the MDAP will be washed with water. We will determine the number of washes in order to completely remove the periodate from the MDAP. The concentration of periodate in the washes can be monitored spectrophotometrically.[B 39] Finally the MDAP will be dried. MDAP can be air dried, but we will also explore the use of an oven or a gel drier to accelerate the drying process.

The degree of oxidation of the MDAP samples will be measured either indirectly, by measuring the consumption of periodate in the reaction solution spectrophotometrically,[B 39] or directly, by measuring the aldehyde content of the MDAP.[B 40] We will look for correlations between the degree of oxidation of MDAP and the other properties of MDAP such as size, mechanical properties and rate of degradation.

Our results indicate that paper can be oxidized to varying degrees by controlling parameters (i.e., periodate concentration and reaction time). We expect to be able to produce a wide variety of MDAPs by changing the initial paper used and the reaction conditions. By analyzing the reaction conditions, we intend to develop a protocol for producing useful MDAPs within 24 h. We expect that this portion of the project will continue throughout the entire project period as we will be developing MDAPs with specific characteristics for specific applications.

Alternative approach: In another approach we can explore the use of other types of paper. In addition to being useful as a scaffold, MDAP may also be useful in point-of-care paper diagnostic devices, where miniaturization could significantly reduce the volume/sample size required for analysis of analyte (data not shown).

### Characterization of MDAP.

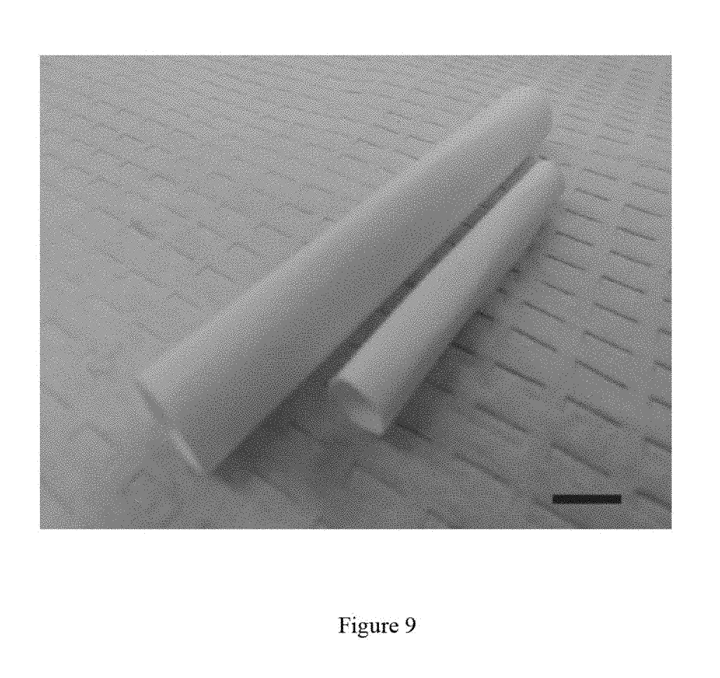

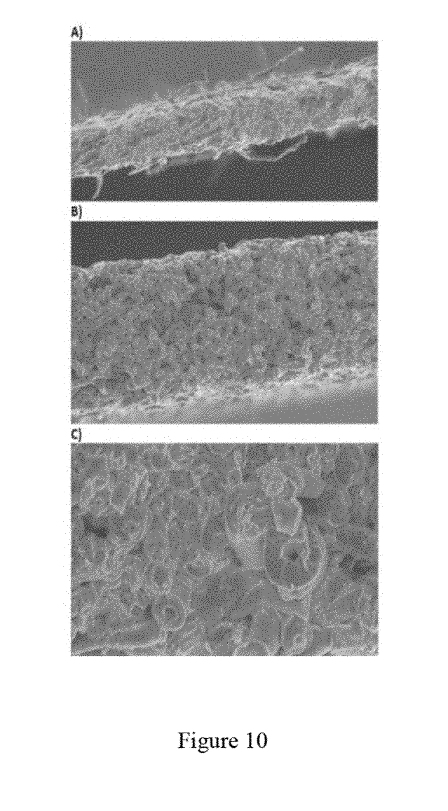

Results obtained indicate that MDAP is malleable and shapeable, while retaining its structural integrity (FIG. 9). Our scanning electron microscopy (SEM) images of MDAP suggest that the miniaturization process results in the ‘swelling’ and ‘hollowing’ of the independent cellulose fibrils (FIG. 10), while at the same time changing the porosity of the paper. Interestingly, the wicking properties of MDAP remain equivalent to non-treated cellulose paper (data not shown), however there is evidence of increased stabilization of enzymatic activity of horseradish peroxidase on MDAP (FIG. 8C). Further characterization of MDAP will be performed in order to determine preferred conditions for preparing MDAP for cell and tissue culture.

### Approach:

For this portion of the project we plan to characterize samples of paper and the corresponding MDAP in terms of wet tensile strength, strain at break, Young's Modulus, morphology, porosity and pore size. We intend to correlate these measurements with the percent-miniaturization and degree of oxidation of the MDAP. Characterization of MDAP will be performed using standard methods and instrumentation that is available through Cal Poly's Materials Engineering Department.[B 39, 40]

### Results:

We predict that we will be able to tune the oxidation reaction and paper in order to produce MDAPs with a range of different properties including the appropriate characteristics for use as scaffolds for culturing different types of cells and tissues.[B 43-45] The degree of oxidation of the paper is expected to affect its rate of degradation in aqueous solution so it will be important to also consider this factor.

### Degradation of MDAP.

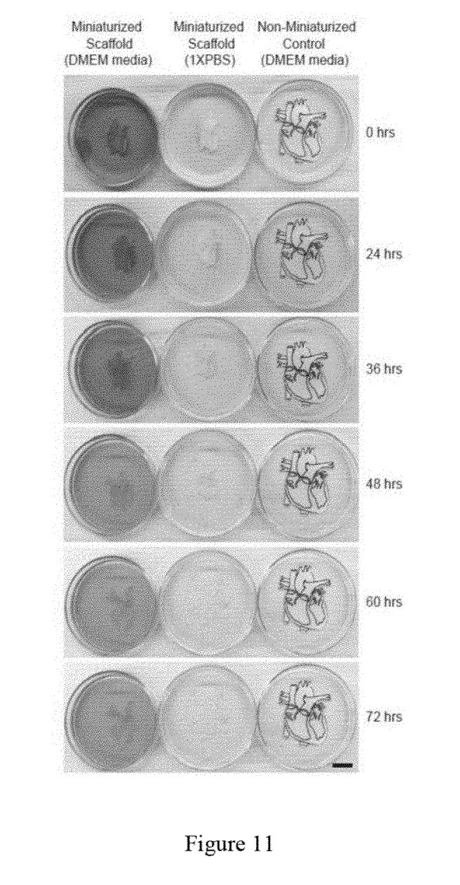

Results indicate that MDAP degrades in aqueous solution (e.g., diH2O, PBS, tissue culture media: Complete DMEM & RPMI) over a period of 72 hrs (FIG. 11). For this portion of the project, our plan is to determine rates of degradation of MDAP as well as the preferred conditions for controlling the degradation of MDAP. Parameters of interest will include: time, temperature, and solution composition and pH.

### Approach:

We will evaluate the rate of degradation of MDAP under conditions of static immersion in a Petri dish as well as in a temperature/humidity controlled continuous-laminar flow chamber that will continuously bathe the MDAP scaffolds with solution at a controllable rate. We will track the progress of the degradation qualitatively by imaging the scaffolds at 6, 12 or 24-hour intervals until a given scaffold is completely degraded. In one set of experiments we will collect the MDAP scaffolds at various time points via filtration, dry them and weigh them in order to quantitatively track the degradation of the scaffolds. We will evaluate the degradation of MDAP in DI water, buffers with pH in the range of 2 to 12, and tissue culture media (DMEM and RPMI) at 4° C., 20° C. and 35-37° C. (the optimal temperature for culturing human cells lines). The flow rate of the solution in the laminar flow chamber will also be varied.

### Results:

We expect that the rate of degradation of MDAP will vary depending on the composition of the MDAP (type of paper and degree of oxidation) and the degradation conditions (solvent, pH, temperature, flow rate). DAC is known to degrade more quickly at higher pH and higher temperatures, so we expect that this will also be the case for MDAP.[B 27, 36] We also expect that MDAP will degrade more quickly in the continuous flow chamber than under static immersion. We expect that by tuning the various parameters, we will be able to achieve degradation of MDAP over a wide range of times from minutes to weeks. This temporal dissolution of a miniaturized scaffold is expected to achieve a tunable scaffold to support the temporal growth of tissue culture and subsequent layers of extra cellular matrix (ECM) deposition (see above).

### Alternative Approach:

We anticipate that the continuous flow of media across the miniaturized device in a standard flow chamber may accelerate the degradation of the MDAP scaffold and/or result in the inappropriate movement of the scaffold for proper monitoring or result in piecemeal degradation of the scaffold due to the inappropriate bridging to its surrounding tissue culture dish. We propose the design and use of plastic cassettes that could affix the MDAP scaffold in place and allow for easier transport and transfer of the device in and out of different bathing conditions.

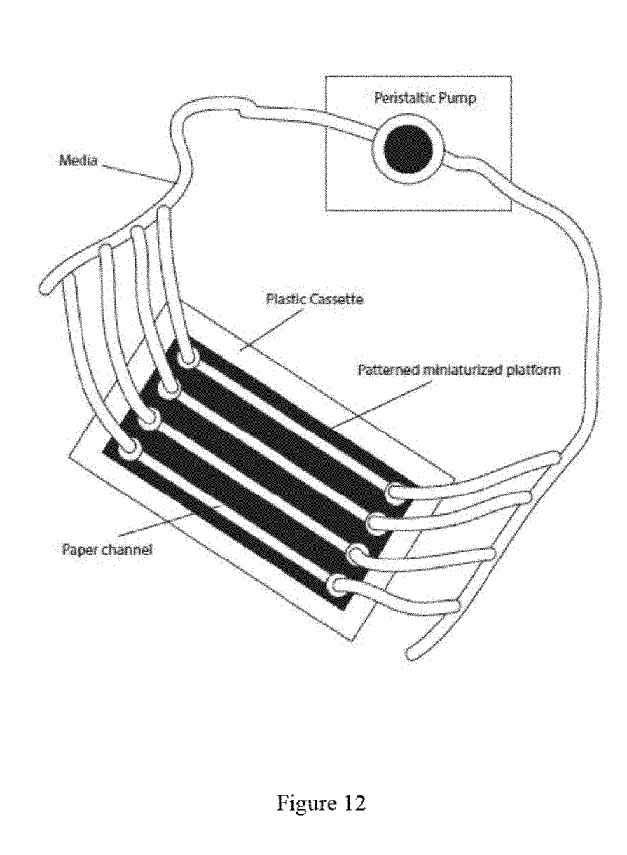

Furthermore, we envision the use of a plastic cassette with micro-channel patterns where differing rates of media and variable conditions can be achieved across patterns on a single device (FIG. 12), thus achieving true temporal control and tunable dissolution of the platform. It is also possible that MDAP scaffolds with the appropriate degradation characteristics may not have the appropriate mechanical properties or vice versa. In order to overcome this potential challenge, we would also investigate the possibility of coating the MDAP scaffolds with additional reagents (e.g., biocompatible polymers) in order to tweak the degradation profile. We will also investigate the effects of patterning the MDAP scaffolds with wax, which may also have a significant impact on the characteristics of the scaffold.

Development of methods for patterning and shaping MDAP into 2D and 3D structures. An important advantage of MDAP as a scaffold is that it can be patterned and shaped into 2D and 3D structures (FIG. 8C, FIG. 9). We will focus primarily on patterning MDAP with wax in order to define hydrophobic barriers on the scaffolds, which in turn will define shapes on the scaffold in 2D. We will also investigate methods of shaping the scaffolds in 3D by either stacking multiple layers of patterned MDAP on top of each other (see above) or by folding the MDAP scaffolds (see above).

### Approach:

Paper can be patterned with wax via a method known as wax printing, [B 46] and then it can be treated with periodate to produce patterned MDAP. We plan to characterize wax-patterned MDAP in more detail to determine the smallest feature sizes that can be produced using this approach and to determine whether the wax patterns affect the properties of MDAP or its rate of degradation. We will also investigate methods of shaping MDAP into 3D structures via stacking, folding and molding. Initial experiments will focus on producing simple structures like open tubes. We will determine whether it is best to shape the paper before treating it with periodate, or after it has been miniaturized. We will also determine whether it is possible to shrink the paper onto 3D scaffolds (made from plastic using a 3D printer) in order to produce more complex shapes. Finally, we will investigate shrinking paper that is first shaped by origami or by cutting/interlocking to produce MDAPs in more complex 3D structures.

### Results:

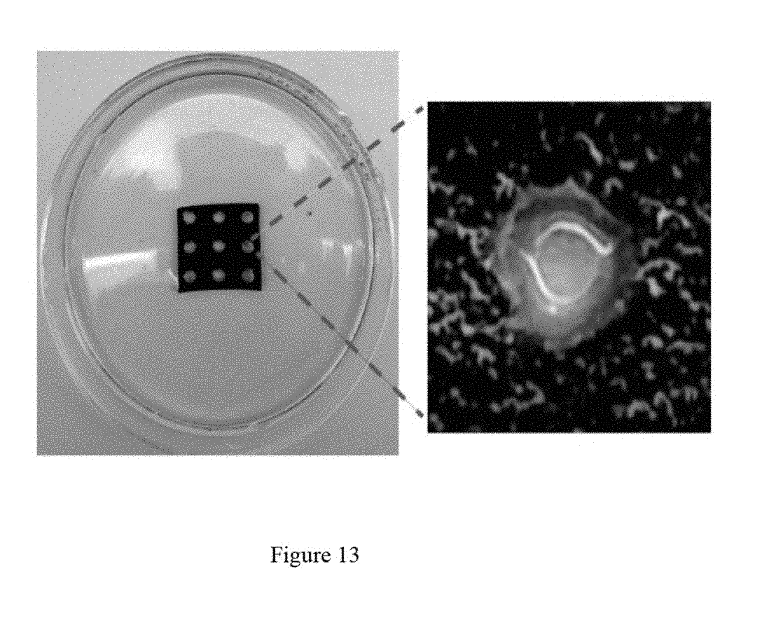

Results indicate that the wax does not interfere with the oxidation reaction (FIGS. 8A and C) and that the wax patterns become miniaturized along with the paper. We have also found that, at least under some conditions, wax-patterned MDAP degrades more slowly compared to wax-free MDAP (FIG. 13), and this may provide a useful approach for preparing arrays of cultured cells or tissues. MDAP is malleable and can be folded or rolled into simple 3D shapes (FIG. 9), but it may be easier to shape the paper before it is oxidized and then miniaturize the shaped paper.

### Alternative Approach:

For certain applications, we believe it will be useful to have patterned MDAP where only the non-patterned areas are degradable, and the patterned areas do not degrade. In order to produce this type of material, we could explore other techniques for patterning paper including the use of a technique for patterning paper with Teflon. [B 1, 47]

### Evaluation of MDAP as a Scaffold for Cell Culture and Tissue Engineering.

Our preliminary data indicate the viability of growing cells under laboratory controlled conditions on MDAP. Briefly, fibroblast-like cells (COS-1 cells) were trypsinized and seeded at 2×10^{\hat{ }5} cells per ml of complete DMEM+10% FBS and then plated in 60 mm tissue culture treated dishes, fitted with wax-patterned MDAP. Cells were incubated at 37° C. for 12 hrs and the patterned MDAP was subsequently transferred to a fresh tissue culture dish.

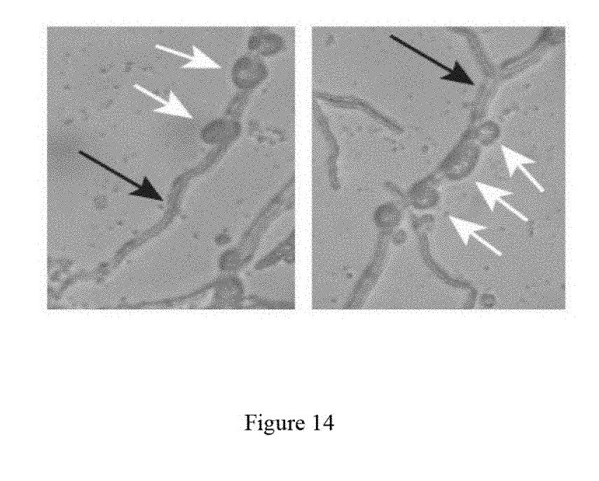

Cell viability and growth was monitored by brightfield microscopy (FIG. 14). This data is in accordance with previously published results demonstrating monolayer cell growth on dialdehyde cellulose scaffolds.[B 39, 48] For this portion of the experiment, we plan to further explore and evaluate MDAP as a scaffold for 3D cell culture and tissue engineering.

### Evaluation of MDAP as a Scaffold for 3D Cell Culture.

For this section of the experiment, our plan is to determine the preferred conditions for growing monolayers of a variety of cell cultures on MDAP, including longstanding laboratory workhorse cell lines such as: COS-1, HeLa & 3T3s, in addition to more specialized cell lines such as: primary human coronary artery endothelial cells (EC), human coronary artery smooth muscle cells (SMC), and primary human coronary artery fibroblasts. Each of these cell cultures will be grown independently to achieve optimal growth parameters. In addition, the comparison of untreated vs. growth factor treated paper will be studied. A major limitation of current tissue engineering scaffolds made from hydrophobic synthetic polymers is inefficient protein loading and limited control of protein release.[B49] We predict the MDAP will display higher protein loading capabilities via adsorption on paper and tunable release time (FIG. 8C) or covalently binding reagents to MDAP.

### Approach:

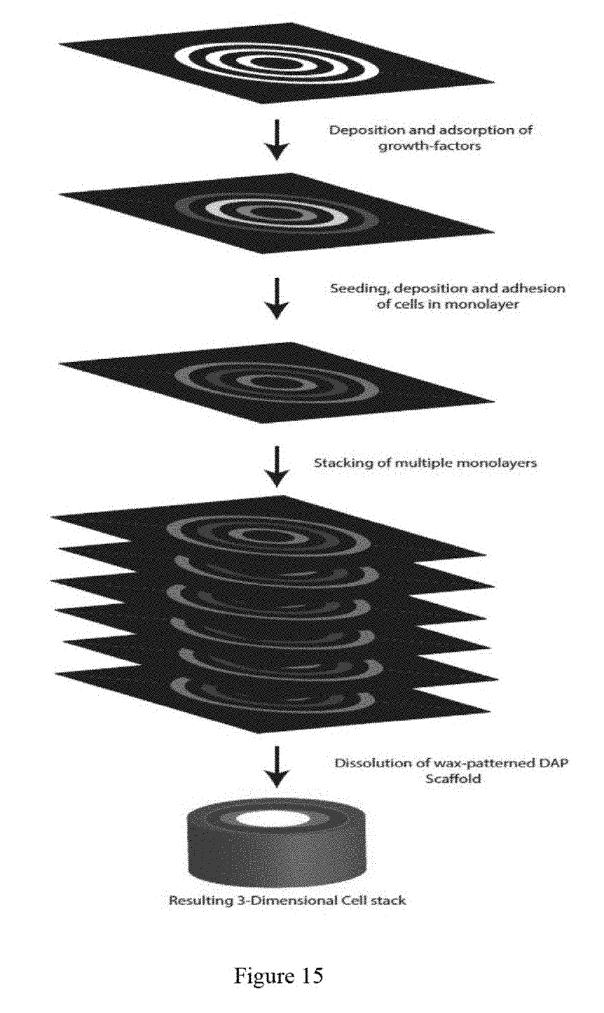

A variety of wax-patterns will be applied onto cellulose paper and miniaturized as described above. After proper sterilization, we will compare growth of cell lines on growth-factor treated and untreated MDAP. Treatment of paper includes the deposition of a mixture of growth-factors (e.g., endothelial cell growth factor (ECGF), vascular endothelial growth factor (VEGF), platelet-derived growth factor (PDGF), and epidermal growth factor (EGF), etc.) that will be applied by micropipette to the wax-free regions of the patterned paper (FIG. 15). Following wicking and adsorption of growth factors, cells will be seeded in growth media and allowed to adhere during incremental incubation time periods (6-24 hrs) (FIG. 15). At these varying time intervals, the paper scaffold will be analyzed for cell adhesion and formation of a monolayer of cells by epifluorescent microscopy and hematoxylin eosin (HE) staining. Scaffold adherent cells will be transferred to fresh tissue culture dishes and allowed to further adhere to the tissue culture dish during dissolution of the scaffold, thus demonstrating transferability and manipulation of the monolayer cell-sheet. Cells will be live stained with Hoechst dye and visualized under an epi-fluorescent microscope to determine cellular arrangement, patterning and viability. In addition, each cell type monolayer will be fluorescently stained with their corresponding cellular surface markers (ECs:CD31/Sca-I, SMCs: SM1/SM2/h-caldesmon) for proper identification. Finally, we will monitor time-dependent collagen and elastin deposition both on the scaffold and then on the tissue culture dish as evidence of ECM deposition. Briefly, collagen will be stained using a 0.1% Picrosirius red solution, washed and de-stained. The absorbance of the resulting solution will be measured at 490 nm and quantified with a spectrophotometer.[B 50] Elastin deposition will be monitored using a modified Verhoff s stain,[B 51] followed by microscopic imaging.

### Results:

Given our initial findings that fibroblast-like cell growth is viable on MDAP scaffolds, we expect that we will achieve growth of transferable monolayer cell-sheets of a variety of cell types in a variety of 2D patterns. We will further develop the MDAP (including its structural and mechanical properties, degradation characteristics, shape and size of patterns, and content of growth factors and other reagents) to achieve optimal and reproducible cell growth. Furthermore, we expect that the rate of MDAP degradation will vary based on cell-type, given differences of ECM deposition and differences in overall protein expression. Growth parameters of each cell type will involve individualized studies.

### Alternative Approaches:

It is well established that primary coronary SMCs are characterized by poor adhesion and spreading and low proliferation.[B 52] We anticipate that the conditions for growth of these different cell types will be varied. Should direct deposition of growth factors on paper not achieve the predicted goals, an alternative approach could include the deposition of several extracellular matrix components (Matrigel®, type I and IV collagen, fibronectin, vitronectin and laminin) directly on the wax-free regions of the miniaturized paper scaffold. This alternative approach has been previously utilized by several groups in an attempt to achieve higher cell adhesion on a variety of polymeric scaffolds.[B53, 54] In addition, we could envision the use of scaffolds for growing a wide variety of non-mammalian cell types, including insect derived cells, plant derived cells and in the study of complex prokaryotic biofilm formation.

### Evaluation of MDAP as a Scaffold for Tissue Culture.

The combination of ease of wax-patterning and the stackable nature of layers of MDAP leads to the conclusion that cells could be readily grown in 3D scaffolds made from patterned MDAP in order to produce viable tissues in defined shapes and sizes.

### Approach:

To obtain 3D growth of cells in a controlled pattern, we propose evaluating two different approaches: 1) culturing monolayers of cells in multiple individual layers of patterned MDAP followed by stacking of the individual layers to produce 3D-structured tissues, and 2) shaping patterned MDAP into 3D structures followed by culturing cells in the shaped scaffold to form the desired tissues.

With stacking MDAP monolayers of cell sheets, we envision exact control of the orientation of growth of each cellular monolayer based on two directions. The first direction is obtained by the inherent directionality of the cellulose fibers and the concomitant attachment of cells. Although there appears to be a degree of heterogeneity to the MDAP fibrils (FIG. 10C), it is presumed that these fibrils have an overall net directionality to them along the x-y plane of the MDAP sheet. Our current observations support the hypothesis of directional growth of cells along MDAP fibrils (FIG. 14). The second direction will be achieved by the printed wax patterns, which will mediate specific 2D parameters and provide defined boundaries for the growth of varying cell types (FIGS. 13 and 15). The ease of wax-printing a variety of patterns should enable us to pattern and grow cells in ways that have not previously been attempted. Finally, a third dimension may be achieved by the stacking of monolayer MDAP cell sheets, resulting in 3-dimensional cell adhesion and growth (FIG. 15). We will start with two layers of MDAP and then expand the work to additional layers. We will determine the limits of this fabrication method by determining the maximum number of layers that can be stacked while still maintaining cell viability.[B 3] This approach will also allow for the creation of complex heterogeneous tissue constructs by layering different types of cells into a wide range of 3D shapes.

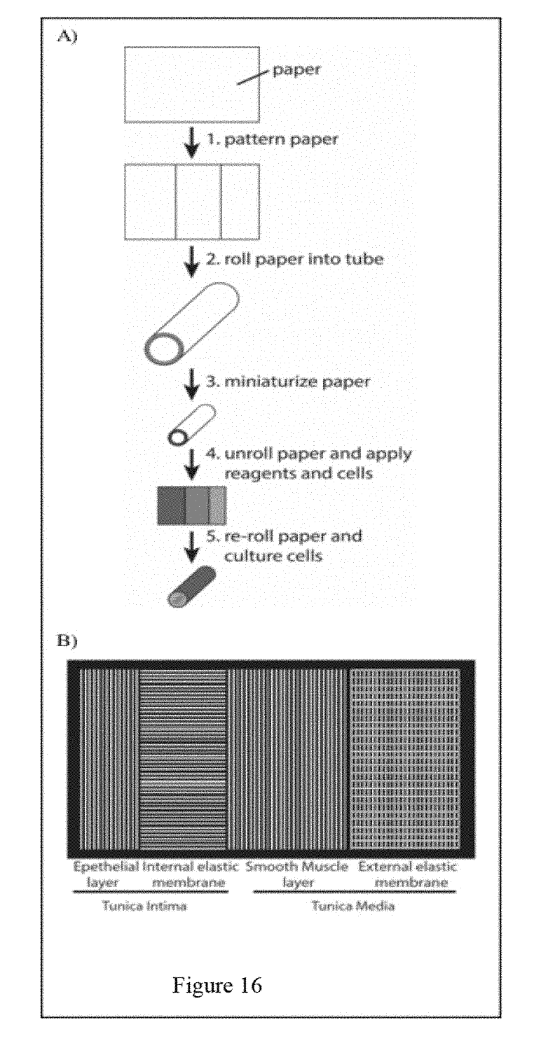

For shaping MDAP into 3D structures prior to cell culture, we propose starting with a simple “jellyroll” structure (FIGS. 9 and 16). A sheet of patterned MDAP will be seeded with growth factors and cells and then rolled into an open-tubular structure. The number of layers of cells in the tube can be easily controlled by tuning the size of the paper and radius of the tube. It should also be possible to use this approach to make stacks of different types of cells in order to create complex heterogeneous tissue constructs.

### Results:

By selecting appropriate cell lines and MDAP scaffolds, we expect to culture 3D-shaped tissues. Preferred conditions for the formation of tissue and for the degradation of the MDAP scaffold will be analyzed. There will likely be a limit to the size of the 3D tissues that can be created using these approaches since the cells in the inner layers of the tissues will need access to nutrients from the media.

### Alternative Approach:

We can test a variety of MDAP scaffolds prepared from different types of paper and to various levels of oxidation. The MDAP scaffolds can also be treated with a variety of reagents and growth factors to induce tissue growth. Combinations of wax-printed, non-wax and non-periodate treated regions can be employed to vary the net stability of the paper scaffold. The scaffolds can also be patterned with holes (produced by cutting the scaffold with a laser cutter) in order to supply media to the cells at the core of 3D tissues.

### MDAP as a Scaffold for Preparing Tissue-Engineered Blood Vessels.

The work with 3D scaffolds will lead directly into a focus on the preparation of tissue-engineered blood vessels (TEBVs). Blood vessels are complex tissues comprising multiple layers of different types of cells.[B 55-57] We will explore different ways of achieving the open-tubular and layered cell structure required for a blood vessel as depicted in FIGS. 15 and 16. Our initial target will be to develop a TEBV with two layers of cells: an outer layer of smooth muscle cells (SMC) and an inner layer of endothelial cells (EC), which has been shown to function as a TEBV in vitro.[B 55] If the techniques prove successful, then more complex TEBV with additional layers of cells could also be explored.

### Approach:

SMCs and ECs will be seeded in patterned MDAP scaffolds as depicted in FIGS. 15 and 16. The cells will be cultured in order to establish a layer of tissue. The MDAP scaffolds will then either be stacked or rolled to form the open-tubular shape of blood vessels. The TEBVs will then be further cultured in vitro to allow the layers of cells to fuse to form a complete vessel. The resulting TEBVs will be tested in vitro by pumping solutions through the vessels to test their ability to withstand the pressures required for arteries.[B 55] It is well established that directionality of blood vessel layers is dependent on directional flow and pressure within the vessel.[B 58] In addition, blood vessels have inherent directionality to their layers (tunica intima is longitudinal, tunica media is circumferential as are the internal and external elastic membranes). We envision using wax patterns to direct the orientation of the layers in the TEBV (FIG. 15B).

### Results:

MDAP scaffolds will provide a new option for preparing TEBVs. We expect that the method of stacking layers of paper will only be suitable for constructing small segments of TEBVs, however, this approach could allow for the incorporation of additional structures (e.g., valves to prevent backflow) in the lumen of the TEBV. The method of rolling the scaffold into a tube should allow for the preparation of longer TEBVs. The open space in the scaffold should allow for the supply of nutrients to the cells in the inner layers of the scaffold.

### Alternative Approaches:

A third approach for forming the tubular structure required for TEBVs is to manufacture paper in the form of a tube and then miniaturize it. It should be possible to prepare multiple concentric tubes that would allow for the seeding of different cell types. The advantage of this approach is that there would be no seams in the tubes, so they may be able to withstand greater pressures.

### Example 4

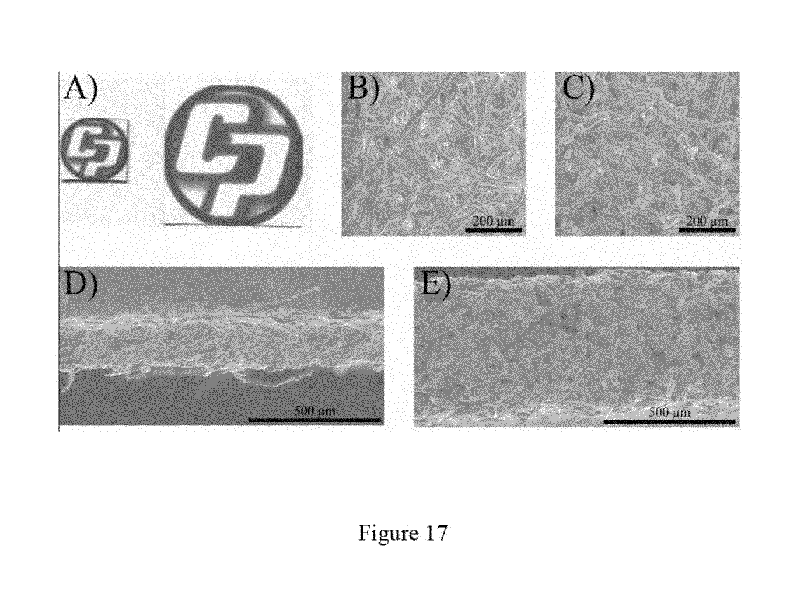

MicroPADs were fabricated on standard cellulose paper (Whatman no. 1) via wax printing. Devices were then submerged in 0.5 M aqueous sodium periodate (NaIO4) for a 48-hour incubation period. Upon extensive rinsing to remove excess periodate ions, devices were desiccated on a slab dryer prior to use (1 hour, 60° C., 300 torr). Functionality of low concentrations of horseradish peroxidase (HRP) (1e-7 M) and HRP conjugated immunoglobulin G (IgG) (1e-7 M) deposited onto microPADs via solvent evaporation were assessed over time. HRP activity was confirmed through a reaction with tetramethylbenzidine (TMB), and analyzed via digital image colorimetry (DIC, ImageJ, NIH). HRP stability was also examined at −20, 4, 25, and 37° C. A three-dimensional (3D) hybrid microPAD was constructed from a combination of both normal and chemically modified cellulose paper. Glucose oxidase, peroxidase, and ABTS were added to the reagent zone via solvent-free deposition [H. T. Mitchell, I. C. Noxon, C. A. Chaplan, et al., “Reagent pencils: a new technique for solvent-free deposition of reagents onto paper-based microfluidic devices,” Lab on a chip, vol. 15, no. 10, pp. 2213-20, 2015]. Hybrid devices were run with 5 mM glucose (sample zone, FIG. 17) and analyzed via DIC.

After saturation in sodium periodate, microPADs displayed a 78% reduction in surface area and a 53% reduction in linear dimensions. Periodate oxidation of cellulose via the Malaprade reaction produces 2,3-dialdehyde cellulose and allows for the reorganization of cellulose fibers into non-linear conformations, ultimately allowing for miniaturization [R. D. Guthrie, “The ‘dialdehydes’ from the periodate oxidation of carbohydrates,” Advances in carbohydrate chemistry, vol. 16, pp. 105-58, 1962]. These miniaturized microPADs were therefore termed miniaturized dialdehyde paper (MDAP).

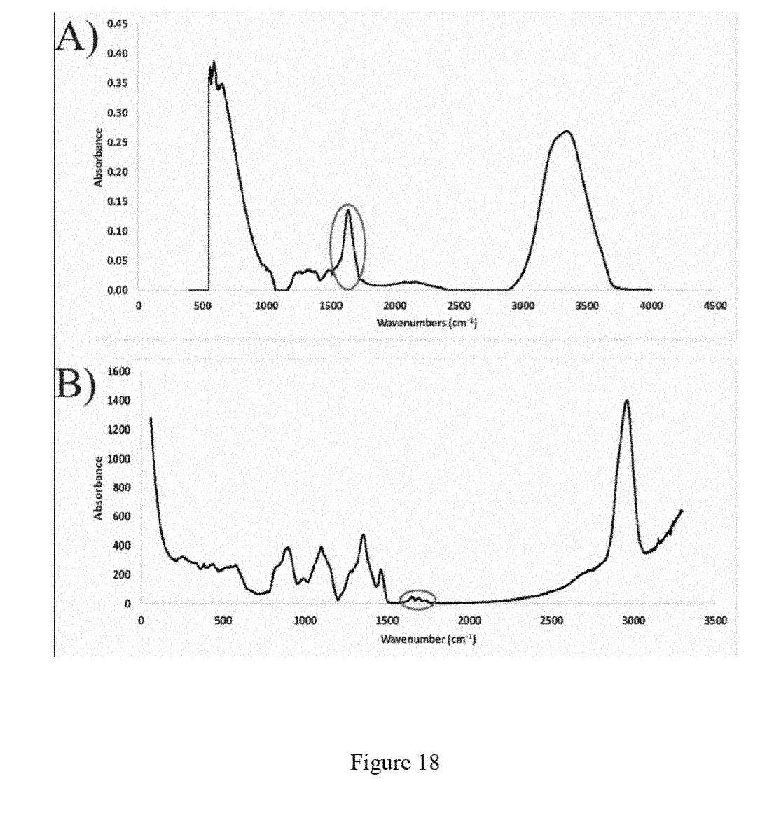

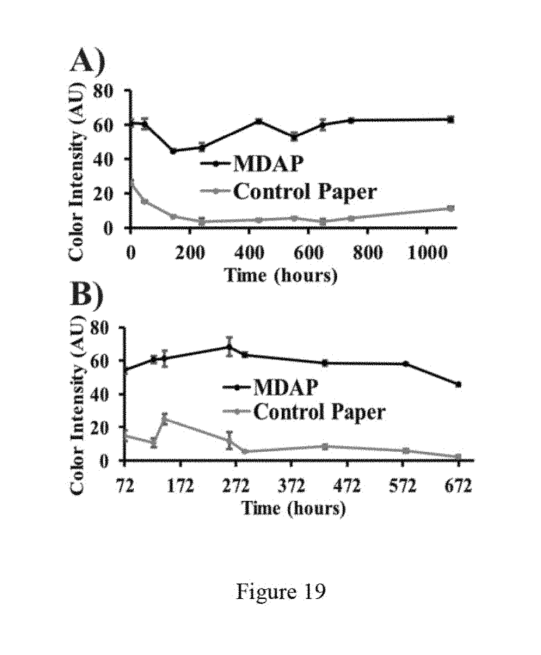

HRP (on MDAP) displayed both immediate (0 hours) and prolonged (1080 hours, 48 days) stabilization as compared to normal cellulose paper (FIG. 18). HRP conjugated to IgG also displayed prolonged enzyme stabilization over a 672-hour (28 day) period (FIG. 17B). Temperature (4, 25, & 37° C.) had no significant effect on enzyme stability after 48 hours (F=2.14, df=2,6, p=0.199), 120 hours (F=1.40, df=2,6, p=0.318), or 168 hours (F=5.15, df=2,6, p=0.050) when stored on MDAP. A three-dimensional MDAP/cellulose hybrid microPAD (FIG. 17) was constructed to display a functionalized form of MDAP. Glucose oxidase (GO) stability was tested on the hybrid device and displayed stability over a 720-hour (30 day) period (FIG. 19). GO functionality declined after 30 days of storage under ambient conditions (FIG. 19). It is important to note that all enzyme stability curves are a function of enzyme concentration.

Without wishing to be bound by any theory, we hypothesize that imine bonds forming between cellulose aldehyde groups and enzyme lysine residues allow for the stabilization of tertiary and quaternary protein structure [F. Lopez-Gallego, L. Betancor, C. Mateo, et al., “Enzyme stabilization by glutaraldehyde crosslinking of adsorbed proteins on animated supports,” Journal of biotechnology, vol. 119, no. 1, pp. 70-75, 2005]. Additionally, the slightly increased hydrophobicity of ¬MDAP, caused by the compaction of cellulose fibers and subsequent reduction in reactivity of hydroxyl groups [R. D. Guthrie, “The ‘dialdehydes’ from the periodate oxidation of carbohydrates,” Advances in carbohydrate chemistry, vol. 16, pp. 105-58, 1962; A. G. Cunha and A. Gandini, “Turning polysaccharides into hydrophobic materials: a critical review,” Cellulose, vol. 17, no. 5, pp. 875-89, 2010.], may also promote enzyme stabilization. While other forms of reagent stability are available [S. Ramachandran, E. Fu, B. Lutz, and P. Yager, “Long-term dry storage of an enzyme-based reagent system for ELISA in point-of-care devices,” Analyst, vol. 139, no. 6, pp. 1456-62, 2014; H. T. Mitchell, I. C. Noxon, C. A. Chaplan, et al., “Reagent pencils: a new technique for solvent-free deposition of reagents onto paper-based microfluidic devices,” Lab on a chip, vol. 15, no. 10, pp. 2213-20, 2015], this method allows for prolonged enzyme functionality when directly applied to microfluidic devices. This could ultimately allow for increased ease-of-use in field or point-of-care settings.

### Example 5

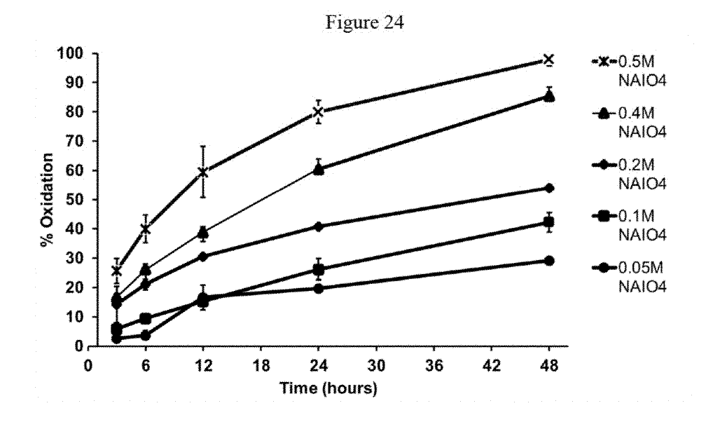

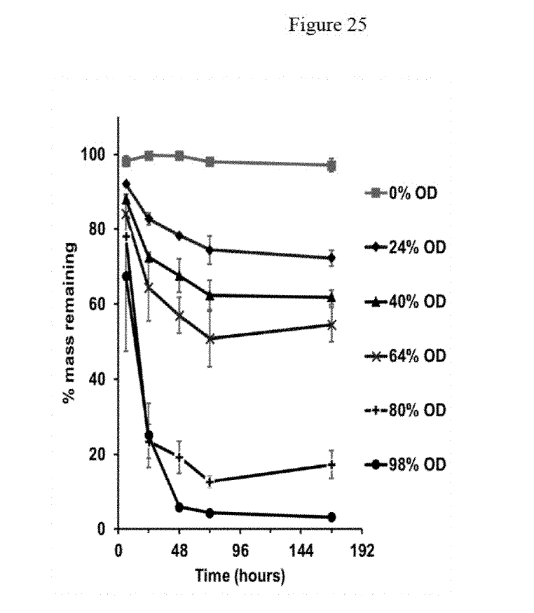

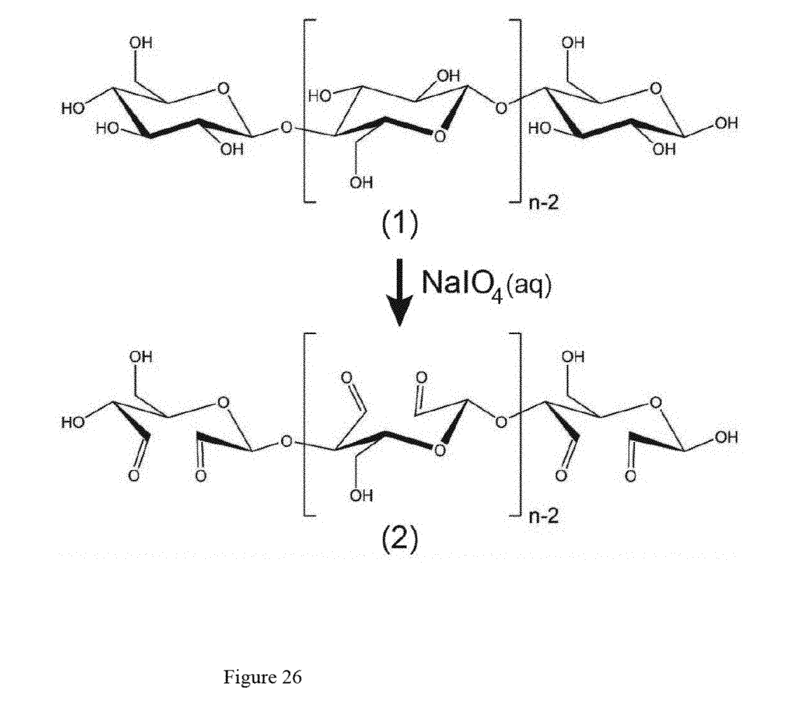

Miniaturization and corresponding degradation of paper scaffolds or supports or a portion has been found to correlate to the percent oxidation of the cellulose fibrils of the paper. See FIGS. 24 and 25. The percent oxidation refers to the percent of glucose sub units in the cellulose that react with periodate (See FIG. 26). This was measured by a titration method referred to as the Cannizzaro method (supra). We show here that preferred embodiments provide oxidation levels of 10% or higher are particularly useful for production of reduced surface area and producing such supports or supports having a portion reduced in surface area.

We further found that miniaturization and the amount of oxidation can be accelerated by performing the reaction at elevated temperatures, such as 25° C. to 95° C. The higher temperatures can also achieve higher concentrations of sodium periodate (0.5-2.5M) which will contribute to a faster reaction rate.

To perform the reaction at 55° C. requires a large test tube (e.g., 25 mm×200 mm), a ring stand, a clamp, a thermometer and a water bath (e.g., a hotplate and 400-mL beaker). A fume hood was used for performing the reaction at temperatures above 55° C. To achieve maximum miniaturization of the paper, the reaction is carried out in a 50-mL centrifuge tube using 40 mL of 0.5-M sodium periodate, prepared using 4.28 g of solid sodium periodate. To significantly increase the speed, the reaction can be carried out in a water bath at higher temperatures (e.g., 55° C.) using 1-M sodium periodate (8.56 g) of sodium periodate dissolved in 40 mL of water. A 1-M concentration of sodium periodate can be achieved at higher temperatures as the solubility of the salt increases. Temperatures above 55° C. lead to even faster reactions, but aqueous solutions of sodium periodate are known to decompose above 55° C., liberating iodine gas[BS1]. Therefore, reactions at elevated temperatures should be conducted in a fume hood, especially if the temperature is above 55° C. At the highest periodate concentration, the reaction takes less than an hour.

### Example 6

In this experiment we achieve ˜90% stabilization of total DNA (as compared to paper and controls) over a 23 week period at RT (i.e. no refrigeration). We also show that we can easily separate the sample by size via gel electrophoresis.

This project proposes a new method of gel electrophoresis sample loading using cellulose-based combs. DNA is pipetted onto and housed in pre-printed wax-formed wells on paper. This paper-based comb can then be directly inserted into a gel and run under the same conditions as standard gel electrophoresis. This new method creates a simpler way of running gel electrophoresis on a day to day basis, as well as laying the foundation for long-term stabilization of DNA.

In addition to the use of standard cellulose combs, this experiment uses the modified cellulose-based material called Miniaturized Dialdehyde Paper (MDAP). As described below, Whatman No. 1 chromatography paper was saturated in 0.5-M NaIO4 and stored in the dark at room temperature for 48 hours. Upon removal from solution, an 80% reduction in surface area and a 166% increase in cross-sectional width is observed. The observed shrinkage is most likely caused by intramolecular hemiacetals between the aldehyde groups and the primary alcohol in 2,3-dialdehyde cellulose. This reaction leads to non-linear conformations and buckling because it cannot occur in a chair conformation, thus causing miniaturization. Additionally, due to increased hydrophobicity of the MDAP, samples are given extended time to dry.

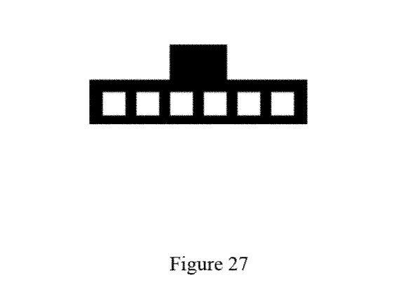

Cellulose paper (Whatman No. 1) was printed with wax to create hydrophobic regions. The wax was printed surrounding 3×2 mm wells, where a DNA sample is pipetted. After printing, the paper was baked in a convection oven at 195° C. for 2 minutes and then allowed to cool to room temperature. Next, to create MDAP, the combs were submerged in a 0.5-M aqueous sodium periodate solution (NaIO4) for 48 hours without exposure to light. Devices were removed from the solution, washed in DI water, and dried on a slab drier (1 hour, 60° C., 300 torr). Upon conclusion of this process, the paper shrank by approximately 80% in surface area, with significant added rigidity. DNA samples were then prepared using the following protocol: 2 μl DNA samples (1 kb ladder) were individually pipetted into each sample well. See FIG. 27. (FIG. 27 is one example of such combs and many iterations are possible.) In addition, a 1% agarose gel was prepared using 0.6 g agarose in 60 ml 1×TAE running buffer. The agarose was dissolved in the buffer by microwaving the solution for 150 s with occasional swirling. Upon removal from the microwave, 2 μl GelRed 10,000× was added to the gel solution. Next, the agarose gel was poured into a sealed gel box, and the MDAP comb was inserted and held in place by taping it to a standard plastic comb. The gel was cooled in a 5° C. fridge until it became solid (approximately 10 minutes).

Running buffer (1×TAE) was then poured onto the solidified gel so that the gel was completely submerged. A negative electrode was connected at the sample end of the gel, and a positive electrode was connected at the opposite end. The gel was run at 200 mV for 3 minutes, after which the MDAP comb was removed. The gel was run for an additional 60 minutes at 110 mV. At the conclusion of the 60 minutes, the gel was removed and imaged using transUV radiation. To determine DNA recovery (ng/ul), a gel extraction was performed. To reduce warping and distortion of DNA bands the wax-printed MDAP/paper comb was removed after a short period of time, in an embodiment, after about three minutes.

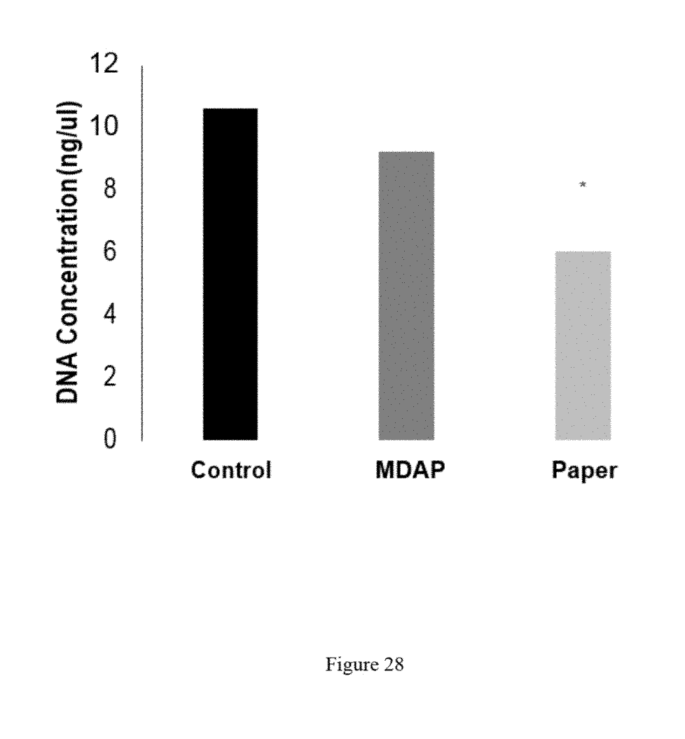

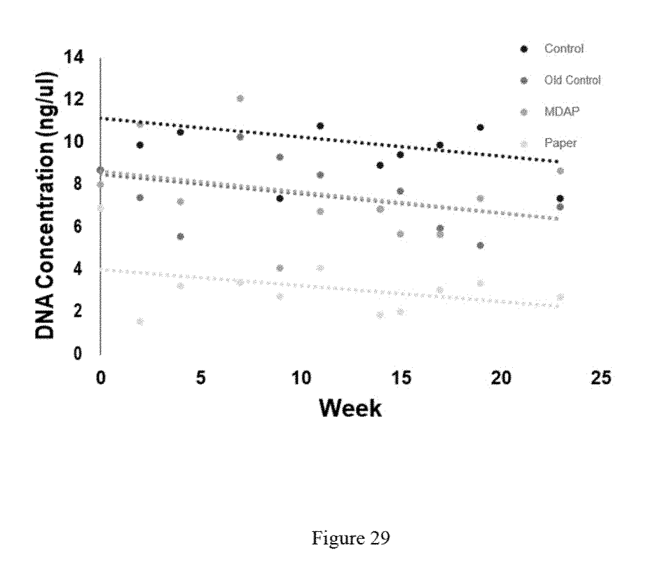

There was a significant difference in the concentration of DNA recovered between Control, MDAP, and Paper combs (F=25.5943, DF=2,165, p<0.0001). There was significantly more DNA recovered from Control wells than Paper wells (p<0.0001), and significantly more DNA recovered from MDAP wells than Paper wells (p<0.0001). There was no significant difference in DNA recovery concentration between Control and MDAP wells (p=0.0920). See FIGS. 28 and 29.

### Example 7

The above experiments are repeated, this time reducing only a portion of the paper support. The support area is contacted with the sodium periodate as described above until the desired reduction in size is achieved. The resulting paper support area is both reduced in size and degradable. In an experiment, wax printed circular zones of about 3 mm radius are created and a peristaltic pump used to deliver warm (about 55° C.) periodate drip-wise to the wax-free cellulose zone.

The foregoing is presented by way of illustration and is not intended to limit the scope of the invention. References cited herein are incorporated herein by reference.

### REFERENCES

1 A. W. Martinez, S. T. Phillips, M. J. Butte and G. M. Whitesides, Angew. Chemie-Int. Ed., 2007, 46, 1318-1320.
2 D. M. Cate, J. A. Adkins, J. Mettakoonpitak and C. S. Henry, Anal. Chem., 2015, 87, 19-41.
3 A. K. Yetisen, M. S. Akram and C. R. Lowe, Lab Chip, 2013, 13, 2210-51.
4 K. Yamada, T. G. Henares, K. Suzuki and D. Citterio, Angew. Chemie—Int. Ed., 2015, 54, 5294-5310.
5 J. Hu, S. Wang, L. Wang, F. Li, B. Pingguan-Murphy, T. J. Lu and F. Xu, Biosens. Bioelectron., 2014, 54, 585-597.
6 E. Carrilho, S. T. Phillips, S. J. Vella, A. W. Martinez and G. M. Whitesides, Anal. Chem., 2009, 81, 5990-5998.
7 R. Derda, A. Laromaine, A. Mammoto, S. K. Y. Tang, T. Mammoto, D. E. Ingber and G. M. Whitesides, Proc. Natl. Acad. Sci., 2009, 106, 18457-18462.
8 R. R. Ravgiala, S. Weisburd, R. Sleeper, A. Martinez, D. Rozkiewicz, G. M. Whitesides and K. A. Hollar, J. Chem. Educ., 2014, 91, 107-111.
9 M. T. Koesdjojo, S. Pengpumkiat, Y. Wu, A. Boonloed, D. Huynh, T. P. Remcho and V. T. Remcho, J. Chem. Educ., 2015, 92, 737-741.
10 A. W. Martinez, S. T. Phillips, G. M. Whitesides and E. Carrilho, Anal. Chem., 2010, 82, 3-10.
11 A. W. Martinez, S. T. Phillips, E. Carrilho, S. W. Thomas, H. Sindi and G. M. Whitesides, Anal. Chem., 2008, 80, 3699-3707.
12 K. Tenda, R. Ota, K. Yamada, T. G. Henares, K. Suzuki and D. Citterio, Micromachines, 2016, 7, 80.
13 A. Grimes, D. N. Breslauer, M. Long, J. Pegan, L. P. Lee and M. Khine, Lab Chip, 2008, 8, 170-2.
14 C.-S. Chen, D. N. Breslauer, J. I. Luna, A. Grimes, W.-C. Chin, L. P. Lee and M. Khine, Lab Chip, 2008, 8, 622-4.
15 D. Nguyen, D. Taylor, K. Qian, N. Norouzi, J. Rasmussen, S. Botzet, M. Lehmann, K. Halverson and M. Khine, Lab Chip, 2010, 10, 1623-1626.
16 A. L. Das, R. Mukherjee, V. Katiyer, M. Kulkarni, A. Ghatak and A. Sharma, Adv. Mater., 2007, 19, 1943-1946.
17 B. Aldalali, A. Kanhere, J. Fernandes, C. C. Huang and H. Jiang, Micromachines, 2014, 5, 275-288.
18 H. M. Fletcher and S. H. Roberts, Text. Res. J., 1953, 23, 37-42.
19 M. Juciene, V. Dobilate and G. Kazlauskaite, Mater. Sci., 2006, 12, 355-359.
20 C. K. F. Hermann, 1997, 74, 1357.
21 E. L. Jackson and C. S. Hudson, J. Am. Chem. Soc., 1937, 10, 2049-2050.
22 L. Malaprade, Bull. Soc. Chim. Fr., 1928, 43, 683-696.
23 L. Malaprade, Bull. Soc. Chim. Fr., 1934, 5, 833-852.
24 U. J. Kim, S. Kuga, M. Wada, T. Okano and T. Kondo, Biomacromolecules, 2000, 1, 488-492.
25 A. Potthast, M. Kostic, S. Schiehser, P. Kosma and T. Rosenau, Holzforschung, 2007, 61, 662-667.
26 T. Morooka, M. Norimoto and T. Yamada, J. Appl. Polym. Sci., 1989, 38, 849-858.
27 R. D. Guthrie, Adv. Carbohydr. Chem., 1962, 16, 105-158.
28 T. P. Nevell, in Cellul. Chem. Its Appl., 1985, pp. 243-265.
29 H. A. Rutherford, F. W. Minor, A. R. Martin and M. Harris, J. Res. Natl. Bur. Stand. (1934), 1942, 29, 131-141.
30 S. Wang, L. Ge, X. Song, M. Yan, S. Ge, J. Yu and F. Zeng, Analyst, 2012, 137, 3821.
31 S. Su, R. Nutiu, C. D. M. Filipe, Y. Li and R. Pelton, Langmuir, 2007, 23, 1300-1302.
32 Y. Xia, J. Si and Z. Li, Biosens. Bioelectron., 2016, 77, 774-789.
33 S. Ahmed, M. P. N. Bui and A. Abbas, Biosens. Bioelectron., 2016, 77, 249-263.
34 G. F. Davidson, J. Text. Inst. Trans., 1941, 32, T109-T131.
35 E. Carrilho, A. W. Martinez and G. M. Whitesides, Anal. Chem., 2009, 81, 7091-7095.
36 R. Lu, W. Shi, L. Jiang, J. Qin and B. Lin, Electrophoresis, 2009, 30, 1497-1500.
37 Y. Lu, W. Shi, J. Qin and B. Lin, Anal. Chem., 2010, 82, 329-335.
38 M. A. Mahmud, E. J. M. Blondeel, M. Kaddoura and B. D. MacDonald, Analyst, 2016, 141, 6449-6454.
39 A. W. Martinez, S. T. Phillips, B. J. Wiley, M. Gupta and G. M. Whitesides, Lab Chip, 2008, 8, 2146-2150.
40 H. T. Mitchell, I. C. Noxon, C. A. Chaplan, S. J. Carlton, C. H. Liu, K. A. Ganaja, N. W. Martinez, C. E. Immoos, P. J. Costanzo and A. W. Martinez, Lab Chip, 2015, 15, 2213-2220.
41 C. H. Liu, I. C. Noxon, L. E. Cuellar, A. L. Thraen, C. E. Immoos, A. W. Martinez and P. J. Costanzo, Micromachines, 2017, 8.
42 C. A. Chaplan, H. T. Mitchell and A. W. Martinez, Anal. Methods, 2014, 6, 1296.
43 E. W. Washburn, Phys. Rev., 1921, 17, 273-283.
44 C. K. Camplisson, K. M. Schilling, W. L. Pedrotti, H. A. Stone and A. W. Martinez, Lab Chip, 2015, 15, 4461-4466.
45 S. Mendez, E. M. Fenton, G. R. Gallegos, D. N. Petsev, S. S. Sibbett, H. A. Stone, Y. Zhang and G. P. López, Langmuir, 2010, 26, 1380-1385.
46 B. M. Cummins, R. Chinthapatla, F. S. Ligler and G. M. Walker, Anal. Chem., 2017, 89, 4377-4381.
47 S. Lathwal and H. D. Sikes, Lab Chip, 2016, 16, 1374-1382.
B1. Yetisen, A. K., Akram, M. S., and Lowe, C. R. “Paper-Based Microfluidic Point-of-Care Diagnostic Devices.” Lab on a chip 13, no. 12 (2013): 2210-51. doi:10.1039/c31c50169h, Available at http://www.ncbi.nlm.nih.gov/pubmed/23652632
B2. Cate, D. M., Adkins, J. A., Mettakoonpitak, J., and Henry, C. S. “Recent Developments in Paper-Based Micro Fluidic Devices” Analytical Chemistry 87, (2015): 19-41. doi:10.1021/ac503968p, Available at http://pubs.acs.org/doi/abs/10.1021/ac503968p
B3. Derda, R., Laromaine, A., Mammoto, A., Tang, S. K. Y., Mammoto, T., Ingber, D. E., and Whitesides, G. M. “Paper-Supported 3D Cell Culture for Tissue-Based Bioassays” Proceedings of the National Academy of Sciences 106, no. 44 (2009): 18457-18462. doi:10.1073/pnas.0910666106, Available at http://www.pnas.org/cgi/doi/10.1073/pnas.0910666106
B4. Merten, O. W. “Introduction to Animal Cell Culture Technology—Past, Present and Future” Cytotechnology 50, no. 1-3 (2006): 1-7. doi:10.1007/s10616-006-9009-4
B5. Philippeos, C., Hughes, R. D., Dhawan, A., and Mitry, R. R. “Introduction to Cell Culture” Methods in Molecular Biology 806, (2012): 1-13. doi:10.1007/978-1-61779-367-7_1
B6. Pollard, J. W. “Basic Cell Culture.” Methods in molecular biology (Clifton, N.J.) 5, (1990): 1-12. doi:10.1385/0-89603-441-0:1
B7. Justice, B. A., Badr, N. A., and Felder, R. A. “3D Cell Culture Opens New Dimensions in Cell-Based Assays” Drug Discovery Today 14, no. 1-2 (2009): 102-107. doi:10.1016/j.drudis.2008.11.006
B8. Huh, D., Hamilton, G. A., and Ingber, D. E. “From 3D Cell Culture to Organs-on-Chips” Trends in Cell Biology 21, no. 12 (2011): 745-754. doi:10.1016/j.tcb.2011.09.005
B9. Langer, R. and Vacanti, J. P. “Tissue Engineering” Science 260, no. 5110 (1993): 920-926. doi:10.1007/978-3-642-02824-3, Available at http://www.scopus.com/inward/record.url?eid=2-s2.0-84857523452&partnerID=tZOtx3y1
B10. Bhat, Z. F., Bhat, H., and Pathak, V. “Principles of Tissue Engineering” Principles of Tissue Engineering (2014): 1663-1683. doi:10.1016/B978-0-12-398358-9.00079-3, Available at http://www.sciencedirect.com/science/article/pii/B9780123983589000793
B11. Chan, B. P. and Leong, K. W. “Scaffolding in Tissue Engineering: General Approaches and Tissue-Specific Considerations” European Spine Journal 17, no. SUPPL. 4 (2008): doi:10.1007/s00586-008-0745-3
B12. Carletti, E., Motta, A., and Migliaresi, C. “Scaffolds for Tissue Engineering and 3D Cell Culture.” Methods in molecular biology (Clifton, N.J.) 695, (2011): 17-39. doi:10.1007/978-1-60761-984-0_2

B13. Hollister, S. J. “Porous Scaffold Design for Tissue Engineering.” Nature materials 4, no. July (2005): 518-24. doi:10.1038/nmat1421, Available at http://www.ncbi.nlm.nih.gov/pubmed/16003400
B14. Modulevsky, D. J., Lefebvre, C., Haase, K., Al-Rekabi, Z., and Pelling, A. E. “Apple Derived Cellulose Scaffolds for 3D Mammalian Cell Culture” PLoS ONE 9, no. 5 (2014): doi:10.1371/journal.pone.0097835
B15. Duan, N. and et al. “A Vascular Tissue Engineering Scaffold with Core-shell Structured Nano-Fibers Formed by Coaxial Electrospinning and Its Biocompatibility Evaluation” Biomedical Materials 11, no. 3 (2016): 35007. doi:10.1088/1748-6041/11/3/035007, Available at http://stacks.iop.org/1748-605X/11/i=3/a=035007?key=crossref.66d0402ba7ec6791 ecf279f46bf201a6
B16. Nandgaonkar, A. G., Krause, W. E., and Lucia, L. A. “Fabrication of Cellulosic Composite Scaffolds for Cartilage Tissue Engineering” Nanocomposites for Musculoskeletal Tissue Regeneration no. October (2016): 187-212. doi:10.1016/B978-1-78242-452-9.00009-1
B17. Chen, G., Ushida, T., and Tateishi, T. “Scaffold Design for Tissue Engineering” Macromolecular Bioscience 2, no. 2 (2002): 67-77. doi:10.1002/1616-5195(20020201)2:2<67::AID-MABI67>3.0.CO;2-F
B18. Martinez, A. W., Phillips, S. T., Whitesides, G. M., and Carrilho, E. “Diagnostics for the Developing World: Microfluidic Paper-Based Analytical Devices” Analytical Chemistry 82, no. 1 (2010): 3-10. doi:10.1021/ac9013989
B19. Park, H. J., Yu, S. J., Yang, K., Jin, Y., Cho, A. N., Kim, J., Lee, B., Yang, H. S., Im, S. G., and Cho, S. W. “Paper-Based Bioactive Scaffolds for Stem Cell-Mediated Bone Tissue Engineering” Biomaterials 35, no. 37 (2014): 9811-9823. doi:10.1016/j.biomaterials.2014.09.002
B20. Kim, S.-H., Lee, H. R., Yu, S. J., Han, M.-E., Lee, D. Y., Kim, S. Y., Ahn, H.-J., Han, M.-J., Lee, T.-I., Kim, T.-S., Kwon, S. K., Im, S. G., and Hwang, N. S. “Hydrogel-Laden Paper Scaffold System for Origami-Based Tissue Engineering” Proceedings of the National Academy of Sciences 112, no. 50 (2015): 201504745. doi:10.1073/pnas.1504745112, Available at http://www.pnas.org/lookup/doi/10.1073/pnas.1504745112
B21. Ng, K., Gao, B., Yong, K. W., Li, Y., Shi, M., Zhao, X., Li, Z., Zhang, X., Pingguan-Murphy, B., Yang, H., and Xu, F. “Paper-Based Cell Culture Platform and Its Emerging Biomedical Applications” Materials Today 0, no. 0 (2016): doi:10.1016/j.mattod.2016.07.001, Available at http://linkinghub.elsevier.com/retrieve/pii/S1369702116300840
B22. Chen, Q., He, Z., Liu, W., Lin, X., Wu, J., Li, H., and Lin, J. M. “Engineering Cell-Compatible Paper Chips for Cell Culturing, Drug Screening, and Mass Spectrometric Sensing” Advanced Healthcare Materials 4, no. 15 (2015): 2291-2296. doi:10.1002/adhm.201500383
B23. Tao, F. F. T., Xiao, X., Lei, K. F., and Lee, I.-C. “Paper-Based Cell Culture Microfluidic System” The Korean BioChip Society and Springer 9, no. 2 (2015): 97-104. doi:10.1007/s13206-015-9202-7
B24. Lei, K. F. and Huang, C. H. “Paper-Based Microreactor Integrating Cell Culture and Subsequent Immunoassay for the Investigation of Cellular Phosphorylation” ACS Applied Materials and Interfaces 6, no. 24 (2014): 22423-22429. doi:10.1021/am506388q
B25. Malaprade, L. “A Study of the Action of Polyalcohols on Periodic Acid and Alkaline Periodates” Bulletin de la Societe Chimique de France 5, no. 1 (1934): 833-852.
B26. Malaprade, L. “Action of Polyalcohols on Periodic Acid. Analytical Application” Bulletin de la Societe Chimique de France 43, (1928): 683-696.
B27. Guthrie, R. D. “THE ‘DIALDEHYDES’ FROM THE PERIODATE OXIDATION OF CARBOHYDRATES” Advances in Carbohydrate Chemistry 16, (1962): 105-158.
B28. Rutherford, H. A., Minor, F. W., Martin, A. R., and Harris, M. “Oxidation of Cellulose: The Reaction of Cellulose with Periodic Acid” Journal of Research of the National Bureau of Standards 29, no. 2 (1942): 131-141.
B29. Perlin, A. S. “Glycol-Cleavage Oxidation” Advances in Carbohydrate Chemistry and Biochemistry 60, (2006): 183-250. doi:10.1016/S0065-2318(06)60005-X
B30. Jackson, E. L. and Hudson, C. S. “Application of the Cleavage Type of Oxidation by Periodic Acid to Starch and Cellulose” Journal of the American Chemical Society 10, no. 59 (1937): 2049-2050.
B31. Nevell, T. P. “Oxidation of Cellulose.” Cellul. Chem. Its Appl. no. 10 (1985): 243-265.
B32. Kim, U. J., Kuga, S., Wada, M., Okano, T., and Kondo, T. “Periodate Oxidation of Crystalline Cellulose.” Biomacromolecules 1, no. 3 (2000): 488-492. doi:10.1021/bm0000337
B33. Sirvio, J., Hyvakko, U., Liimatainen, H., Niinimaki, J., and Hormi, O. “Periodate Oxidation of Cellulose at Elevated Temperatures Using Metal Salts as Cellulose Activators” Carbohydrate Polymers 83, no. 3 (2011): 1293-1297. doi:10.1016/j.carbpol.2010.09.036
B34. Davidson, G. F. “The Progressive Oxidation of Cotton Cellulose By Periodic Acid and Metaperiodate Over a Wide Range of Oxygen Consumption” Journal of the Textile Institute Transactions 32, no. 7 (1941): T109-T131. doi:10.1080/19447024108659362, Available at http://www.tandfonline.com/doi/abs/10.1080/19447024108659362
B35. Mack, C. H. and Reeves, W. A. “Wrinkle Resistant Properties of Dialdehyde Cotton” Textile Research Journal 31, no. 9 (1961): 800-803.
B36. Kim, U. J., Wada, M., and Kuga, S. “Solubilization of Dialdehyde Cellulose by Hot Water” Carbohydrate Polymers 56, no. 1 (2004): 7-10. doi:10.1016/j.carbpol.2003.10.013
B37. Potthast, A., Schiehser, S., Rosenau, T., and Kostic, M. “Oxidative Modifications of Cellulose in the Periodate System—Reduction and Beta-Elimination Reactions: 2nd ICC 2007, Tokyo, Japan, Oct. 25-29, 2007” Holzforschung 63, no. 1 (2009): 12-17. doi:10.1515/HF.2009.108
B38. Singh, M., Ray, A. R., and Vasudevan, P. “Biodegradation Studies on Periodate Oxidized Cellulose” Biomaterials 3, no. 1 (1982): 16-20. doi:10.1016/0142-9612(82)90055-2
B39. RoyChowdhury, P. and Kumar, V. “Fabrication and Evaluation of Porous 2,3-Dialdehydecellulose Membrane as a Potential Biodegradable Tissue-Engineering Scaffold” Journal of Biomedical Materials Research—Part A 76, no. 2 (2006): 300-309. doi:10. 1002/jbm.a.30503
B40. Li, J., Wan, Y., Li, L., Liang, H., and Wang, J. “Preparation and Characterization of 2,3-Dialdehyde Bacterial Cellulose for Potential Biodegradable Tissue Engineering Scaffolds” Materials Science and Engineering C 29, no. 5 (2009): 1635-1642. doi:10. 1016/j.msec.2009.01.006, Available at http://dx.doi.org/10.1016/i.msec.2009.01.006
B41. Nery, E. W. and Kubota, L. T. “Evaluation of Enzyme Immobilization Methods for Paper-Based Devices-A Glucose Oxidase Study” Journal of Pharmaceutical and Biomedical Analysis 117, (2016): 551-559. doi:10.1016/j.jpba.2015.08.041
B42. “Whatman Product Guide” (2011):
B43. Shearn, J. T., Juncosa-Melvin, N., Boivin, G. P., Galloway, M. T., Goodwin, W., Gooch, C., Dunn, M. G., and Butler, D. L. “Mechanical Stimulation of Tendon Tissue Engineered Constructs: Effects on Construct Stiffness, Repair Biomechanics, and Their Correlation.” Journal of biomechanical engineering 129, no. 6 (2007): 848-54. doi:10.1115/1.2800769, Available at http://www.ncbi.nlm.nih.gov/pubmed/18067388
B44. Backdahl, H., Helenius, G., Bodin, A., Nannmark, U., Johansson, B. R., Risberg, B., and Gatenholm, P. “Mechanical Properties of Bacterial Cellulose and Interactions with Smooth Muscle Cells” Biomaterials 27, no. 9 (2006): 2141-2149. doi:10. 1016/j.biomaterials.2005.10.026
B45. Nirmalanandhan, V. S., Dressier, M. R., Shearn, J. T., Juncosa-Melvin, N., Rao, M., Gooch, C., Bradica, G., and Butler, D. L. “Mechanical Stimulation of Tissue Engineered Tendon Constructs: Effect of Scaffold Materials.” Journal of biomechanical engineering 129, no. 6 (2007): 919-23. doi:10.1115/1.2800828, Available at http://www.ncbi.nlm.nih.gov/pubmed/18067397
B46. Carrilho, E., Martinez, A. W., and Whitesides, G. M. “Understanding Wax Printing: A Simple Micropatterning Process for Paper-Based Microfluidics” Analytical Chemistry 81, no. 16 (2009): 7091-7095. doi:10.1021/ac901071p
B47. Deiss, F., Matochko, W. L., Govindasamy, N., Lin, E. Y., and Derda, R. “Flow-through Synthesis on Teflon-Patterned Paper to Produce Peptide Arrays for Cell-Based Assays” Angewandte Chemie—International Edition 53, no. 25 (2014): 6374-6377. doi:10. 1002/anie.201402037
B48. Novotna, K., Pavel, H., Tomas, S., Kolarova, K., Vosmanska, V., Lisa, V., Svorcik, V., and Bavakova, L. “Cellulose-Based Materials as Scaffolds for Tissue Engineering” Cellulose 20, no. 5 (2013): 2263-2278. Available at http://link.springer.com/article/10.1007/s10570-013-0006-4
B49. Jay, S. M., Shepherd, B. R., Bertram, J. P., Pober, J. S., and Saltzman, W. M. “Engineering of Multifunctional Gels Integrating Highly Efficient Growth Factor Delivery with Endothelial Cell Transplantation” The FASEB Journal 22, no. 8 (2008): 2949-2956. doi:10.1096/fj.08-108803, Available at http://www.fasebj.org/content/22/8/2949%5Cnhttp://www.fasebi.org/content/22/8/2949.full.pdf %5Cnhttp://www.fasebj.org/content/22/8/2949.long%5Cnhttp://www.ncbi.nlm.nih.gov/pubmed/18450813
B50. Delaine-Smith, R. M., Green, N. H., Matcher, S. J., MacNeil, S., and Reilly, G. C. “Monitoring Fibrous Scaffold Guidance of Three-Dimensional Collagen Organisation Using Minimally-Invasive Second Harmonic Generation” PLoS ONE 9, no. 2 (2014): doi:10.1371/journal.pone.0089761
B51. Abcam. “ab150667 Elastic (Connective Tissue Stain)” (2013):
B52. Patel, S., Shi, Y., Niculescu, R., Chung, E. H., Martin, J. L., and Zalewski, A. “Characteristics of Coronary Smooth Muscle Cells and Adventitial Fibroblasts.” Circulation 101, no. 5 (2000): 524-32. doi:10.1161/01.cir.101.5.524, Available at http://www.ncbi.nlm.nih.gov/pubmed/10662750
B53. Hein, M., Fischer, J., Kim, D. K., and Pratt, R. E. “Vascular Smooth Muscle Cell Phenotype Influences Glycosaminoglycan Composition and Growth Effects of Extracellular Matrix” Journal of Vascular Research 33, no. 6 (1996): 433-441.
B54. Thyberg, J. and Hultgardh-Nilsson, A. “Fibronectin and the Basement Membrane Components Laminin and Collagen Type IV Influence the Phenotypic Properties of Subcultured Rat Aortic Smooth Muscle Cells Differently” Cell and Tissue Research 276, no. 2 (1994): 263-271. doi:10.1007/BF00306112
B55. Huang, A. H. and Niklason, L. E. “Engineering of Arteries in Vitro” Cellular and Molecular Life Sciences 71, no. 11 (2014): 2103-2118. doi:10.1007/s00018-013-1546-3
B56. L'Heureux, N., McAllister, T. N., and la Fuente, L. M. de. “Tissue-Engineered Blood Vessel for Adult Arterial Revascularization” The New England Journal of Medicine 357, no. (2007): 1451-1453. doi:10.1056/NEJMcO71536, Available at http://www.nejm.org/doi/full/10.1056/NEJMc071536%5Cnhttp://www.ncbi.nlm.nih.gov/pubmed/17914054
B57. Zhang, W. J., Liu, W., Cui, L., and Cao, Y. “Tissue Engineering of Blood Vessel.” Journal of cellular and molecular medicine 11, no. 5 (2007): 945-57. doi:10.1111/j.1582-4934.2007.00099.x, Available at http://www.pubmedcentral.nih.gov/articlerender.fcgi?artid=4401266&tool=pmcentrez&rendertype=abstract
B58. L'Heureux, N., Dusserre, N., Konig, G., Victor, B., Keire, P., Wight, T. N., Chronos, N. A. F., Kyles, A. E., Gregory, C. R., Hoyt, G., Robbins, R. C., and McAllister, T. N. “Human Tissue-Engineered Blood Vessels for Adult Arterial Revascularization.” Nature medicine 12, no. 3 (2006): 361-5. doi:10.1038/nm1364, Available at http://www.ncbi.nlm.nih.gov/pubmed/16491087%5Cnhttp://www.pubmedcentral.nih.gov/articlerender.fcgi?artid=PMC1513140
B59. Prof. Y M. “Elastica Light” Available at https://www.pinterest.com/pin/454793262340272302/
B60. Prof Y M. “Spherical Dome & Ellipsoid Dome” Available at https://www.pinterest.com/pin/457396905884583737/
B61. June. “Origami X-Wing” Available at http://images.google.de/imgres?imgurl=http%3A%2F%2Fwww.planetjune.com%2Fblog%2Fimages%2Forigami_x-wing.jpg&imgrefurl=http%3A%2F%2Fwww.planetjune.com%2Fblog%2Forigami-x-wing%2F&h=289&w=450&tbnid=KIOanBO2mMu4bM%3A&docid=cnhVbkvTp_bKMM&ei=RCAXWJ71G8regAayh4zIDA&tbm=isch&iact=rc&uact=3&dur=529&page=0& start=0& ndsp=33&ved=0ahUKEwiep_KA7oTQAhVKL8AKHbIDA8kQMwgiKAYwBg&bih=755&biw=1536

What is claimed is:

1. A paper support for fluid or cells, comprising a paper support wherein said support or portion thereof comprises reduced surface area after contact with a composition comprising sodium periodate compared to surface area of said support or portion thereof when not contacted with said composition comprising sodium periodate and said support or portion thereof having reduced surface area is degradable.

2. The support of claim 1, wherein said reduced surface area of said support or portion thereof is reduced by at least 10%.

3. The support of claim 1, wherein cellulose of said support or portion thereof contacted with said composition comprising sodium periodate is oxidized by at least 10%.

4. The support of claim 3, wherein said oxidation of said cellulose is 10% to 100%.

5. The support of claim 1, wherein said composition comprising sodium periodate comprises at least 0.1M sodium periodate.

6. The support of claim 1, wherein said composition comprising sodium periodate is maintained at a temperature of 20° C. to 95° C.

7. The support of claim 1, wherein the entire support comprises a dialdehyde paper support having reduced surface area and wherein said dialdehyde paper support is degradable.

8. The support of claim 7, wherein said support comprises hydrophilic channels or hydrophobic barriers or both.

9. The support of claim 8, wherein said hydrophobic barriers comprise wax.

10. The support of claim 7, wherein said support prior to contact with said composition comprising sodium periodate has a predetermined shape, said reduced surface area paper support having the same predetermined shape after contact with said composition comprising sodium periodate.

11. The support of claim 10, wherein said reduced surface area paper support is malleable.

12. The support of claim 1, wherein said support comprises a protein and/or nucleic acid molecule in said reduced surface area, said protein and/or nucleic acid molecule having the same or increased stability compared to said support or portion thereof comprising said protein when not contacted with said composition comprising sodium periodate.

13. The support of claim 12, wherein said protein and/or nucleic acid molecule is selected from the group consisting of enzymes, DNA and RNA.

14. The support of claim 1, wherein said reduced surface area paper support or portion thereof is degradable by an aqueous solution.

15. A method of producing a paper support for fluid or cells said support of a portion thereof having reduced surface area, the method comprising, contacting said support or portion thereof with a composition comprising sodium periodate for a period of time until said support or portion thereof surface area is reduced compared to said support or portion thereof when not exposed to said composition comprising sodium periodate and producing a paper support wherein said support or portion thereof has reduced surface area and is degradable.

16. The method of claim 15, wherein said composition is contacted with said support or portion thereof until the percent of oxidation of cellulose of said paper contacted with said composition is reduced by at least 10%.

17. The method of claim 16, wherein said oxidation comprises 10% to 100%.

18. The method of claim 15, wherein said composition is maintained at a temperature of 20° C. to 95° C. and said composition comprises at least 2M sodium periodate.

19. The method of claim 17, wherein the entire support surface area is reduced.

20. A method of improving use of a paper support, the method comprising, contacting a support or a portion thereof with a composition comprising sodium periodate for a period of time until the surface area of said support or a portion thereof is decreased compared to said support or a portion thereof when not contacted with said composition comprising sodium periodate, to produce a support or portion thereof comprising dialdehyde cellulose, wherein:
i) said support has reduced surface area and has decreased volume of fluid necessary to fill said support compared to said support when not contacted with said composition comprising sodium periodate; or
ii) said support has reduced surface area and has decreased average wicking velocity compared to said paper support when not contacted with said composition comprising sodium periodate; or
iii) said support of portion thereof further comprises a protein and/or nucleic acid molecule, said protein and/or nucleic acid molecule having increased stability compared said paper support or portion thereof comprising said protein when not contacted with said composition comprising sodium periodate; or
iv) a combination of i)-iv),
thereby improving use of said paper support.
